(19) United States
(12) Patent Application Publication
(10) Pub. No.: US 2026/0087709 A1
(43) Pub. Date: Mar. 26, 2026

(54) GENERATION OF SYNTHETIC RADIOLOGICAL IMAGES

(71) Applicant: BAYER AKTIENGESELLSCHAFT, Leverkusen (DE)

(72) Inventors: Matthias LENGA, Leverkusen (DE); Ivo Matteo BALTRUSCHAT, Leverkusen (DE); Felix Karl KREIS, Leverkusen (DE)

(21) Appl. No.: 19/107,368

(22) PCT Filed: Aug. 23, 2023
(86) PCT No.: PCT/EP2023/073104

§ 371 Date: Feb. 27, 2025

(30) Foreign Application Priority Data
Aug. 30, 2022 (EP) 22192907.8
Mar. 1, 2023 (EP) 23159288.2
Mar. 1, 2023 (EP) 23159289.0

(51) Int. Cl. G06T 11/00; A61B 6/00; A61K 49/10; G06T 7/00; G16H 30/40

(52) U.S. Cl. CPC G06T 12/20; A61B 6/481; A61B 6/5247; A61K 49/108; G06T 7/0014; G16H 30/40; G06T 2207/10081; G06T 2207/10088; G06T 2207/20081; G06T 2207/20084; G06T 2207/30056; G06T 2210/41; G06T 2211/441

(57) ABSTRACT

The present invention relates to the technical field of radiology. The invention relates to a new approach for training a machine learning model for generating synthetic radiological images on the basis of measured radiological images and to the use of the trained machine learning model for generating synthetic radiological images.

### TECHNICAL FIELD

The present disclosure relates to the technical field of radiology. The subjects of the present disclosure are a novel approach for training a machine-learning model for generation of synthetic radiological images on the basis of measured radiological images and the use of the trained machine-learning model for generation of synthetic radiological images.

### INTRODUCTION

Medical imaging is the technique and process of imaging the interior of a body for clinical analysis and medical intervention, and visual representation of the function of some organs or tissues. Medical imaging seeks to reveal internal structures hidden by the skin and bones and to diagnose and/or treat disease.

Advances in imaging and in machine learning have led to a rapid increase in the potential use of artificial intelligence for various tasks in medical imaging, for example for risk assessment, detection, diagnosis, prognosis and therapy.

Machine-learning models are used for, inter alia, segmenting radiological images, enhancing contrasts in radiological images and/or predicting a radiological image in a temporal sequence of radiological images.

WO2019/074938A1 discloses, for example, a method for reducing the amount of contrast agent in the generation of radiological images with the aid of an artificial neural network.

WO2021/052896A1 discloses a method in which an MRI image (MRI: magnetic resonance imaging) of the liver of a patient during the hepatobiliary phase is not generated by measurement, but is instead calculated (predicted) on the basis of MRI images from one or more preceding phases in order to shorten the time spent by the patient in the MRI scanner.

WO2021/197996A1 discloses a method in which MRI images after the administration of a so-called blood-pool contrast agent are simulated with the aid of a machine-learning model.

The following publications disclose methods for generating an artificial CT image (CT: computed tomography) on the basis of a measured MRI image: M. Maspero et al.: Dose evaluation of fast synthetic-CT generation using a generative adversarial network for general pelvis MR-only radiotherapy, Physics in Medicine and Biology 63(18), 185001; H. Yang et al.: Unsupervised MR-to-CT Synthesis Using Structure-Constrained CycleGAN, IEEE Transactions on Medical Imaging 39(12), 4249-4261.

The following publications discloses methods for generating an artificial T2-weighted MRI image on the basis of a measured T1-weighted MRI image: X. Liu et al.: A Unified Conditional Disentanglement Framework for Multimodal Brain MR Image Translation (June 2021). https://doi.org/10.48550/arXiv.2101.05434; L. Shen et al.: Multi-Domain Image Completion for Random Missing Input Data, IEEE Transactions on Medical Imaging 40(4), 1113-1122.

In all of the aforementioned methods, at least one measured radiological image of an examination region of an examination object is fed to a trained machine-learning model and the model generates a synthetic radiological image. Both the fed radiological image and the synthetic radiological image are real-space representations of the examination region.

It has been found that the synthetic radiological images often contain artifacts. Either fine structures may not be correctly reproduced, or regions of the synthetic radiological image may contain structures not found in the represented tissue (see for example: K. Schwarz et al.: On the Frequency Bias of Generative Models, https://doi.org/10.48550/arXiv.2111.02447).

### SUMMARY

This and other problems are addressed by the present disclosure.

The present disclosure provides in a first aspect a computer-implemented method for training a machine-learning model. The training method comprises the steps of:
receiving and/or providing training data, the training data comprising a set of input data and target data for each examination object of a multiplicity of examination objects,
each set comprising
   at least one input representation of an examination region of the examination object in a first state as input data and
   a target representation of the examination region of the examination object in a second state and a transformed target representation as target data, the transformed target representation representing at least part of the examination region of the examination object in a different space compared to the target representation,
training a machine-learning model, the machine-learning model being configured to generate on the basis of at least one input representation of an examination region of an examination object and model parameters a synthetic representation of the examination region of the examination object,
wherein the training comprises for each examination object of the multiplicity of examination objects:
   feeding the at least one input representation of the examination region of the examination object to the machine-learning model,
   receiving a synthetic representation of the examination region of the examination object from the machine-learning model,
   generating and/or receiving a transformed synthetic representation on the basis of the synthetic representation and/or in relation to the synthetic representation, the transformed synthetic representation representing at least part of the examination region of the examination object in a different space compared to the synthetic representation,
   quantifying the differences i) between at least part of the synthetic representation and at least part of the target representation and ii) between at least part of the transformed synthetic representation and at least part of the transformed target representation by means of a loss function,
   reducing the differences by modifying model parameters,
outputting and/or storing the trained machine-learning model and/or the model parameters and/or transmitting the trained machine-learning model and/or the model parameters to a separate computer system and/or using the trained machine-learning model for generation of a synthetic representation of the examination region of an examination object, preferably a new examination object.

The present disclosure further provides a computer-implemented method (prediction method) for generating a synthetic radiological image with the aid of the trained machine-learning model. The prediction method comprises the steps of
providing a trained machine-learning model,
   the trained machine-learning model having been trained by means of training data to generate on the basis of at least one input representation of an examination region of an examination object in a first state a synthetic representation of the examination region in a second state,
   the training data comprising for each examination object of a multiplicity of examination objects i) at least one input representation of the examination region, ii) a target representation of the examination region and iii) a transformed target representation,
   the at least one input representation representing the examination region of the examination object in the first state and the target representation representing the examination region of the examination object in the second state,
   the transformed target representation representing at least part of the examination region of the examination object in a different space compared to the target representation,
   the training of the machine-learning model comprising reducing differences between i) at least part of the synthetic representation and at least part of the target representation and ii) between at least part of a transformed synthetic representation and at least part of the transformed target representation,
receiving at least one input representation of an examination region of a new examination object in the first state,
inputting the at least one input representation of the examination region of the new examination object into the trained machine-learning model,
receiving a synthetic representation of the examination region of the new examination object in the second state from the machine-learning model,
outputting and/or storing the received synthetic representation and/or transmitting the received synthetic representation to a separate computer system.

The present disclosure further provides a computer system comprising
a receiving unit,
a control and calculation unit and
an output unit,
   wherein the control and calculation unit is configured to cause the receiving unit to receive at least one input representation of an examination region of a new examination object in a first state,
   wherein the control and calculation unit is configured to input the received at least one input representation into a trained machine-learning model,
      the trained machine-learning model having been trained by means of training data to generate on the basis of at least one input representation of an examination region of an examination object in a first state a synthetic representation of the examination region in a second state,
      the training data comprising for each examination object of a multiplicity of examination objects i) at least one input representation of the examination region, ii) a target representation of the examination region and iii) a transformed target representation,
      the at least one input representation representing the examination region of the examination object in the first state and the target representation representing the examination region of the examination object in the second state,
      the transformed target representation representing at least part of the examination region of the examination object in a different space compared to the target representation,
      the training of the machine-learning model comprising reducing differences between i) at least part of the synthetic representation and at least part of the target representation and ii) between at least part of a transformed synthetic representation and at least part of the transformed target representation,
   wherein the control and calculation unit is configured to receive from the machine-learning model a synthetic representation of the examination region of the new examination object in the second state,
   wherein the control and calculation unit is configured to cause the output unit to output the received synthetic representation and/or to store it and/or to transmit it to a separate computer system.

The present disclosure further provides a computer program product comprising a computer program which can be loaded into a working memory of a computer system, where it causes the computer system to execute the following steps:
providing a trained machine-learning model,
   the trained machine-learning model having been trained by means of training data to generate on the basis of at least one input representation of an examination region of an examination object in a first state a synthetic representation of the examination region in a second state,
   the training data comprising for each examination object of a multiplicity of examination objects i) at least one input representation of the examination region, ii) a target representation of the examination region and iii) a transformed target representation,
   the at least one input representation representing the examination region of the examination object in the first state and the target representation representing the examination region of the examination object in the second state,
   the transformed target representation representing at least part of the examination region of the examination object in a different space compared to the target representation,
   the training of the machine-learning model comprising reducing differences between i) at least part of the synthetic representation and at least part of the target representation and ii) between at least part of a transformed synthetic representation and at least part of the transformed target representation,
receiving at least one input representation of an examination region of a new examination object in the first state,
inputting the at least one input representation of the examination region of the new examination object into the trained machine-learning model,
receiving a synthetic representation of the examination region of the new examination object in the second state from the machine-learning model,
outputting and/or storing the received synthetic representation and/or transmitting the received synthetic representation to a separate computer system.

The present disclosure further provides for the use of a contrast agent in a radiological examination method, the radiological examination method comprising:
providing a trained machine-learning model,
   the trained machine-learning model having been trained by means of training data to generate on the basis of at least one input representation of an examination region of an examination object in a first state a synthetic representation of the examination region of the examination region in a second state,
   the training data comprising for each examination object of a multiplicity of examination objects i) at least one input representation of the examination region, ii) a target representation of the examination region and iii) a transformed target representation,
   the at least one input representation representing the examination region of the examination object in the first state and the target representation representing the examination region of the examination object in the second state,
   the transformed target representation representing at least part of the examination region of the examination object in a different space compared to the target representation,
   the training of the machine-learning model comprising reducing differences between i) at least part of the synthetic representation and at least part of the target representation and ii) between at least part of a transformed synthetic representation and at least part of the transformed target representation,
receiving at least one input representation of an examination region, the at least one input representation representing the examination region in the first state before and/or after the administration of the contrast agent,
inputting the at least one input representation of the examination region of the new examination object into the trained machine-learning model,
receiving a synthetic representation of the examination region of the new examination object from the machine-learning model, the synthetic representation representing the examination region in the second state before and/or after the administration of the contrast agent,
outputting and/or storing the received synthetic representation and/or transmitting the received synthetic representation to a separate computer system.

The present invention further provides a contrast agent for use in a radiological examination method, the radiological examination method comprising:
providing a trained machine-learning model,
   the trained machine-learning model having been trained by means of training data to generate on the basis of at least one input representation of an examination region of an examination object in a first state a synthetic representation of the examination region in a second state,
   the training data comprising for each examination object of a multiplicity of examination objects i) at least one input representation of the examination region, ii) a target representation of the examination region and iii) a transformed target representation,
   the at least one input representation representing the examination region of the examination object in the first state and the target representation representing the examination region of the examination object in the second state,
   the transformed target representation representing at least part of the examination region of the examination object in a different space compared to the target representation,
   the training of the machine-learning model comprising reducing differences between i) at least part of the synthetic representation and at least part of the target representation and ii) between at least part of a transformed synthetic representation and at least part of the transformed target representation,
receiving at least one input representation of an examination region, the at least one input representation representing the examination region in the first state before and/or after the administration of the contrast agent,
inputting the at least one input representation of the examination region of the new examination object into the trained machine-learning model,
receiving a synthetic representation of the examination region of the new examination object from the machine-learning model, the synthetic representation representing the examination region in the second state before and/or after the administration of the contrast agent,
outputting and/or storing the received synthetic representation and/or transmitting the received synthetic representation to a separate computer system.

The present disclosure further provides a kit comprising a contrast agent and a computer program product comprising a computer program which can be loaded into a working memory of a computer system, where it causes the computer system to execute the following steps:
providing a trained machine-learning model,
   the trained machine-learning model having been trained by means of training data to generate on the basis of at least one input representation of an examination region of an examination object in a first state a synthetic representation of the examination region in a second state,
   the training data comprising for each examination object of a multiplicity of examination objects i) at least one input representation of the examination region, ii) a target representation of the examination region and iii) a transformed target representation,
   the at least one input representation representing the examination region of the examination object in the first state and the target representation representing the examination region of the examination object in the second state,
   the transformed target representation representing at least part of the examination region of the examination object in a different space compared to the target representation,
   the training of the machine-learning model comprising reducing differences between i) at least part of the synthetic representation and at least part of the target representation and ii) between at least part of a transformed synthetic representation and at least part of the transformed target representation,
receiving at least one input representation of an examination region, the at least one input representation representing the examination region in the first state before and/or after the administration of the contrast agent,
inputting the at least one input representation of the examination region of the new examination object into the trained machine-learning model,
receiving a synthetic representation of the examination region of the new examination object from the machine-learning model, the synthetic representation representing the examination region in the second state before and/or after the administration of the contrast agent,
outputting and/or storing the received synthetic representation and/or transmitting the received synthetic representation to a separate computer system.

Further subjects and embodiments can be found in the following description, the claims and the drawings.

### BRIEF DESCRIPTION OF THE DRAWINGS

FIG. 1 shows by way of example and in schematic form the process of training the machine-learning model.

FIG. 2 shows by way of example and in schematic form a use of a trained machine-learning model for prediction.

FIG. 3 shows by way of example and in schematic form a further embodiment of the training of the machine-learning model.

FIG. 4 shows by way of example and in schematic form a further embodiment of the training of the machine-learning model.

FIG. 5 shows by way of example and in schematic form a further embodiment of the training of the machine-learning model.

FIG. 6 shows by way of example and in schematic form a further embodiment of the training of the machine-learning model.

FIG. 7 shows by way of example and in schematic form a further embodiment of the training of the machine-learning model.

FIG. 8 shows by way of example and in schematic form a further embodiment of the training of the machine-learning model.

FIG. 9 shows by way of example and in schematic form a further use of a trained machine-learning model for prediction.

FIG. 10 shows by way of example and in schematic form a further embodiment of the training of the machine-learning model.

FIG. 11 shows by way of example and in schematic form a further embodiment of the training of the machine-learning model.

FIG. 12 shows by way of example and in schematic form a further use of a trained machine-learning model for prediction.

FIG. 13 shows the result of validation (a) of the machine-learning model trained according to FIG. 10 and (b) of the machine-learning model trained according to FIG. 11.

FIG. 14 shows by way of example and in schematic form a computer system according to the present disclosure.

FIG. 15 shows by way of example and in schematic form a further embodiment of the computer system according to the present disclosure.

FIG. 16 shows schematically in the form of a flow chart one embodiment of the method for training a machine-learning model.

FIG. 17 shows schematically in the form of a flow chart one embodiment of the method for generating a synthetic representation of an examination region of an examination object with the aid of the trained machine-learning model.

### DETAILED DESCRIPTION

The invention will be more particularly elucidated below without distinguishing between the subjects of the invention (training method, prediction method, computer system, computer program product, use, contrast agent for use, kit). Rather, the following remarks are intended to apply mutatis mutandis to all subjects of the invention (training method, prediction method, computer system, computer program product, use, contrast agent for use, kit), irrespective of the context in which the remarks are made.

Where steps are stated in an order in the present description or in the claims, this does not necessarily mean that the invention is limited to the order stated. Instead, it is conceivable that the steps are also executed in a different order or else in parallel with one another, the exception being when one step builds on another step, thereby making it imperative that the step building on the previous step be executed next (which will however become clear in the individual case). The orders stated are thus preferred embodiments of the invention.

In certain places the invention will be more particularly elucidated with reference to drawings. The drawings show specific embodiments having specific features and combinations of features, which are intended primarily for illustrative purposes; the invention is not to be understood as being limited to the features and combinations of features shown in the drawings. Furthermore, statements made in the description of the drawings in relation to features and combinations of features are intended to be generally applicable, that is to say applicable to other embodiments too and not limited to the embodiments shown.

With the aid of the present invention, representations of an examination region of an examination object can be predicted. Such a predicted representation is also referred to as a synthetic representation in this disclosure.

The “examination object” is normally a living being, preferably a mammal, most preferably a human.

The “examination region” is part of the examination object, for example an organ or part of an organ, such as the liver, brain, heart, kidney, lung, stomach, intestines, pancreas, thyroid gland, prostate, breast or part of the aforementioned organs, or multiple organs or another part of the body.

In one embodiment, the examination region includes a liver or part of a liver or the examination region is a liver or part of a liver of a mammal, preferably a human.

In a further embodiment, the examination region includes a brain or part of a brain or the examination region is a brain or part of a brain of a mammal, preferably a human.

In a further embodiment, the examination region includes a heart or part of a heart or the examination region is a heart or part of a heart of a mammal, preferably a human.

In a further embodiment, the examination region includes a thorax or part of a thorax or the examination region is a thorax or part of a thorax of a mammal, preferably a human.

In a further embodiment, the examination region includes a stomach or part of a stomach or the examination region is a stomach or part of a stomach of a mammal, preferably a human.

In a further embodiment, the examination region includes a pancreas or part of a pancreas or the examination region is a pancreas or part of a pancreas of a mammal, preferably a human.

In a further embodiment, the examination region includes a kidney or part of a kidney or the examination region is a kidney or part of a kidney of a mammal, preferably a human.

In a further embodiment, the examination region includes one or both lungs or part of a lung of a mammal, preferably a human.

In a further embodiment, the examination region includes a breast or part of a breast or the examination region is a breast or part of a breast of a female mammal, preferably a female human.

In a further embodiment, the examination region includes a prostate or part of a prostate or the examination region is a prostate or part of a prostate of a male mammal, preferably a male human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

A “representation of the examination region” is normally the result of a radiological examination.

“Radiology” is the branch of medicine that is concerned with the use of predominantly electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present invention thus encompasses in particular the following examination methods: computed tomography, magnetic resonance imaging, sonography.

In a preferred embodiment of the present invention, the radiological examination is a magnetic resonance imaging examination or computed tomography examination.

Computed tomography (CT) is an imaging method for depicting body structures by means of X-radiation. Typically, a rotating X-ray tube revolves around the usually recumbent examination object. The X-rays penetrate the examination region and are attenuated to varying degrees according to the density of the tissue in the different organs. Tissue with high density (for example bone tissue) usually appears as light regions in the images, whereas tissue with low density usually appears dark.

Magnetic resonance imaging, MRI for short, is an imaging method that is used especially in medical diagnostics for depicting structure and function of tissues and organs in the human or animal body.

In MRI, the magnetic moments of protons in an examination object are aligned in a basic magnetic field, with the result that there is a macroscopic magnetization along a longitudinal direction. This is then deflected from the resting position by irradiation with high-frequency (HF) pulses (excitation). The return of the excited states to the resting position (relaxation), or magnetization dynamics, is then detected as relaxation signals by means of one or more HF receiver coils.

For spatial encoding, rapidly switched magnetic gradient fields are superimposed on the basic magnetic field. The captured relaxation signals, or detected MRI data, are initially in the form of raw data in frequency space (so-called k-space data), and can be transformed by subsequent inverse Fourier transform into real space (image space).

Contrast agents are commonly used in radiological examination methods. “Contrast agents” are substances or mixtures of substances that improve the depiction of structures and functions of the body in radiological examinations.

Examples of contrast agents can be found in the literature (see for example A. S. L. Jascinth et al.: Contrast Agents in computed tomography: A Review, Journal of Applied Dental and Medical Sciences, 2016, Vol. 2, Issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M. R. Nough et al.: Radiographic and magnetic resonances contrast agents: Essentials and tips for safe practices, World J Radiol. 2017 Sep. 28; 9(9): 339-349; L. C. Abonyi et al.: Intravascular Contrast Media in Radiography: Historical Development & Review of Risk Factors for Adverse Reactions, South American Journal of Clinical Research, 2016, Vol. 3, Issue 1, 1-10; ACR Manual on Contrast Media, 2020, ISBN: 978-1-55903-012-0; A. Ignee et al.: Ultrasound contrast agents, Endosc Ultrasound. 2016 November-December; 5(6): 355-362).

In one embodiment of the present disclosure, at least one input representation of the at least one input representation and/or the target representation and/or the synthetic representation represents the examination region after the administration of a contrast agent.

In a preferred embodiment, the contrast agent is an MRI contrast agent (irrespective of whether it is used in a magnetic resonance tomography examination method or in a computed tomography method).

The MRI contrast agent may be an extracellular contrast agent. Extracellular contrast agents refer to low-molecular-weight, water-soluble compounds that, after intravenous administration, are distributed to the blood vessels and the interstitial spaces. After a certain, comparatively short period of circulation in the bloodstream, they are excreted via the kidneys. Extracellular MRI contrast agents include, for example, the gadolinium chelates gadobutrol (Gadovist®), gadoteridol (Prohance®), gadoteric acid (Dotarem®), gadopentetic acid (Magnevist®) and gadodiamide (Omnican®).

The MRI contrast agent may be an intracellular contrast agent. Intracellular contrast agents are taken up into the cells of tissues to a certain extent and subsequently excreted. Intracellular MRI contrast agents based on gadoxetic acid are distinguished for example in that they undergo a degree of specific uptake by liver cells (hepatocytes), accumulate in the functional tissue (parenchyma) and enhance contrasts in healthy liver tissue, before being subsequently excreted via the gallbladder into the feces. Examples of such contrast agents based on gadoxetic acid are described in U.S. Pat. No. 6,039,931A; they are commercially available for example under the trade names Primovist® and Eovist®. A further MRI contrast agent having a lower uptake into the hepatocytes is gadobenate dimeglumine (Multihance®).

Gadoxetate disodium (GD, Primovist®) belongs to the group of intracellular contrast agents. It is authorized for use in MRI of the liver for detecting and characterizing lesions in patients with known or suspected focal liver disease. With its lipophilic ethoxybenzyl unit, GD exhibits two-phase spreading: spreading at first in the intravascular and interstitial space after bolus injection, followed by selective uptake by hepatocytes. GD is excreted from the body unaltered via the kidneys and the hepatobiliary route (50:50 dual mechanism of excretion) in about the same amounts. Because of its selective accumulation in healthy liver tissue, GD is also referred to as a hepatobiliary contrast agent.

GD is approved at a dose of 0.1 ml/kg body weight (BW) (0.025 mmol/kg BW Gd). The recommended administration of GD comprises an undiluted intravenous bolus injection at a flow rate of about 2 ml/second, followed by flushing of the i.v. cannula with a physiological saline solution. A standard protocol for liver imaging using GD consists of multiple planning and pre-contrast sequences. After i.v. bolus injection of the contrast agent, dynamic images are usually acquired during the arterial phase (about 30 seconds after the injection, p.i.), portal venous phase (about 60 seconds p.i.) and transitional phase (about 2-5 minutes p.i.). Typically, the transitional phase already shows a certain rise in the liver signal intensity owing to the incipient uptake of the agent into hepatocytes. Additional T2-weighted and diffusion-weighted (DWI) images can be generated after the dynamic phase and before the late hepatobiliary phase.

In one embodiment, the contrast agent is gadoxetate disodium.

In one embodiment of the present disclosure, the contrast agent is an agent that includes gadolinium(III) 2-[4,7,10-tris(carboxymethyl)-1,4,7,10-tetrazacyclododec-1-yl]acetic acid (also referred to as gadolinium-DOTA or gadoteric acid).

In a further embodiment, the contrast agent is an agent that includes gadolinium(III) ethoxybenzyldiethylenetriaminepentaacetic acid (Gd-EOB-DTPA); preferably, the contrast agent includes the disodium salt of gadolinium(III) ethoxybenzyldiethylenetriaminepentaacetic acid (also referred to as gadoxetic acid).

In one embodiment of the present disclosure, the contrast agent is an agent that includes gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate (also referred to as gadopiclenol) (see for example WO2007/042504 and WO2020/030618 and/or WO2022/013454).

In one embodiment of the present disclosure, the contrast agent is an agent that includes dihydrogen [(±)-4-carboxy-5,8,11-tris(carboxymethyl)-1-phenyl-2-oxa-5,8,11-triazatridecan-13-oato(5-)]gadolinate(2-) (also referred to as gadobenic acid).

In one embodiment of the present disclosure, the contrast agent is an agent that includes tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57(10): 629-638; WO2016193190).

In one embodiment of the present disclosure, the contrast agent is an agent that includes a Gd³⁺ complex of a compound of the formula (I)

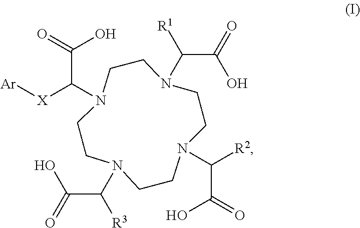

where
Ar is a group selected from

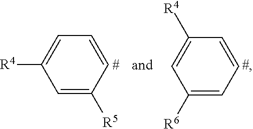

where _{#} is the linkage to X,
X is a group selected from
CH₂, (CH₂)₂, (CH₂)₃, (CH₂)₄ and *—(CH₂)₂—O—CH₂-^{#},
where * is the linkage to Ar and ^{#} is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof.

In one embodiment of the present disclosure, the contrast agent is an agent that includes a Gd³⁺ complex of a compound of the formula (II)

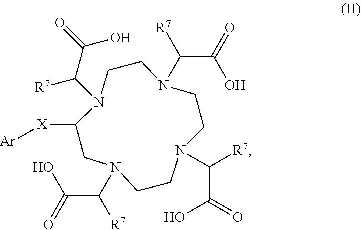

where
Ar is a group selected from

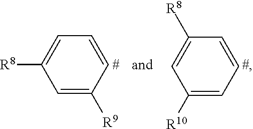

where _{#} is the linkage to X,
X is a group selected from CH₂, (CH₂)₂, (CH₂)₃, (CH₂)₄ and *—(CH₂)₂—O—CH₂-^{#}, where * is the linkage to Ar and ^{#} is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

The term “C₁-C₃ alkyl” denotes a linear or branched, saturated monovalent hydrocarbon group having 1, 2 or 3 carbon atoms, for example methyl, ethyl, n-propyl or isopropyl. The term “C₂-C₄ alkyl” denotes a linear or branched, saturated monovalent hydrocarbon group having 2, 3 or 4 carbon atoms.

The term “C₂-C₄ alkoxy” denotes a linear or branched, saturated monovalent group of the formula (C₂-C₄ alkyl)-O—, in which the term “C₂-C₄ alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate) (see for example WO2022/194777, example 15).

In one embodiment of the present disclosure, the contrast agent is an agent that includes gadolinium 2,2′,2″-{10-[(1S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium 2,2′,2″-{(2S)-10-(carboxymethyl)-2-[4-(2-ethoxyethoxy)benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium(III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxoethyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

A representation of the examination region in the context of the present disclosure may be an MRI image, a CT image, an ultrasound image or the like, or the representation of the examination region may be generated from one or more MRI images, CT images, ultrasound images or the like.

A representation of the examination region may for the purposes of the present disclosure be a representation in real space (image space), a representation in frequency space, a representation in projection space or a representation in another space.

In a representation in real space, also referred to in this description as real-space depiction or real-space representation, the examination region is normally represented by a large number of image elements (pixels or voxels) that may for example be in a raster arrangement, in which case each image element represents a part of the examination region and each image element may be assigned a color value or gray value. The color value or gray value represents a signal intensity, for example the attenuation of X-rays. A format widely used in radiology for storing and processing representations in real space is the DICOM format. DICOM (Digital Imaging and Communications in Medicine) is an open standard for storing and exchanging information in medical image data management.

In a representation in frequency space, also referred to in this description as frequency-space depiction or frequency-space representation, the examination region is represented by a superposition of fundamental frequencies. For example, the examination region may be represented by a sum of sine and cosine functions having different amplitudes, frequencies and phases. The amplitudes and phases may be plotted as a function of the frequencies, for example, in a two- or three-dimensional representation. Normally, the lowest frequency (origin) is placed in the center. The further away from this center, the higher the frequencies. Each frequency can be assigned an amplitude representing the frequency in the frequency-space depiction and a phase indicating the extent of the shift of the respective wave with respect to a sine or cosine wave.

A representation in real space can for example be converted (transformed) by a Fourier transform into a representation in frequency space. Conversely, a representation in frequency space can for example be converted (transformed) by an inverse Fourier transform into a representation in real space.

Details about real-space depictions and frequency-space depictions and their respective interconversion are described in numerous publications, see for example https://see.stanford.edu/materials/lsoftaee261/book-fall-07.pdf.

A representation of an examination region in projection space is normally the result of a computed tomography examination prior to image reconstruction. A projection-space depiction can be understood as meaning raw data in the computed tomography examination. In computed tomography, the intensity or attenuation of X-radiation as it passes through the examination object is measured. From this, projection values can be calculated. In a second step, the object information encoded by the projection is transformed into an image (real-space depiction) through a computer-aided reconstruction. The reconstruction can be effected with the Radon transform. The Radon transform describes the link between the unknown examination object and its associated projections.

Details about the transformation of projection data into a real-space depiction are described in numerous publications, see for example K. Fang: The Radon Transformation and Its Application in Tomography, Journal of Physics Conference Series 1903(1):012066.

A representation of the examination region can also be a representation in Hough space. Geometric objects in an image are detected by performing edge detection followed by what is known as the Hough transform to create a dual space containing all possible parameters of the geometric object for each point in the image that lies on an edge. Each point in dual space accordingly corresponds to a geometric object in image space. For a straight line this can be for example the slope and the y-intercept of the straight line and for a circle this can be the center and radius of the circle. Details about the Hough transform can be found in the literature (see for example A. S. Hassanein et al.: A Survey on Hough Transform, Theory, Techniques and Applications, arXiv: 1502.02160v1).

Representations and/or transformed representations of the examination region are possible in other spaces. It has been found that a machine-learning model can make better predictions if said machine-learning model is not only trained to predict a representation in one space (e.g. in real space), but is also trained to predict a representation of the examination region in a different space (e.g. in frequency space and/or in projection space).

With the aid of a trained machine-learning model, it is possible to generate on the basis of at least one representation of an examination region of an examination object in a first state a synthetic representation of the examination region of the examination object in a second state. The at least one representation, on the basis of which the (trained) machine-learning model generates the synthetic representation, is also referred to in this description as an input representation.

The synthetic representation normally represents the same examination region of the same examination object as the at least one input representation, on the generation of which the synthetic representation is based. The input representation represents the examination region in a first state; the synthetic representation represents the examination region in a second state. The first state and the second state are different states.

For example, the synthetic representation may, compared to the input representation, represent the examination region at a different moment and/or in a different period of time and/or with a different amount of contrast agent and/or with a different contrast agent and/or with a different contrast distribution and/or different contrast agent distribution and/or in segmented form and/or as the result of a different radiological examination method and/or as the result of a radiological examination method based on a different examination protocol (measurement protocol).

In one embodiment, the machine-learning model is configured and trained to generate, on the basis of at least one input representation of an examination region of an examination object representing the examination region before and/or after the administration of a first amount of contrast agent, a synthetic representation representing the examination region after the administration of a second amount of contrast agent, the first amount being preferably lower than the second amount. Such a trained machine-learning model may be used, for example, for reducing the amount of contrast agent in radiological examinations, as described in WO2019/074938A1. Such a trained machine-learning model may be used for converting a radiological image generated after the administration of a first (lower) amount of a contrast agent into a radiological image which, with regard to contrast distribution, has the appearance of a radiological image after the administration of a second (larger) amount of the contrast agent. In other words, the machine-learning model may be trained to produce contrast enhancement without the need to increase the amount of contrast agent.

In a further embodiment, the machine-learning model is configured and trained to generate, on the basis of at least one input representation of an examination region of an examination object representing the examination region in a first period of time before and/or after the administration of an amount of a contrast agent, a synthetic representation representing the examination region in a second period of time after the administration of the amount of the contrast agent. Such a trained machine-learning model may be used, for example, for reducing the length of time that has to be spent by an examination object in an MRI or CT scanner, as described in WO2021/052896A1.

In a further embodiment, the machine-learning model is configured and trained to generate, on the basis of at least one input representation of an examination region of an examination object representing the examination region before and/or after the administration of an amount of a first contrast agent, a synthetic representation representing the examination region after the administration of an amount of a second contrast agent, the first contrast agent and the second contrast agent being different. Such a trained machine-learning model may be used, for example, for simulating MRI images after the administration of a so-called blood-pool contrast agent, as described in WO2021/197996A1.

In a further embodiment, the machine-learning model is configured and trained to generate, on the basis of at least one input representation of an examination region of an examination object representing the examination region as the result of a first radiological examination (e.g. MRI or CT), a synthetic representation representing the examination region as the result of a second radiological examination (e.g. CT or MRI), the first radiological examination and the second radiological examination being different.

In a further embodiment, the machine-learning model is configured and trained to generate, on the basis of at least one input representation of an examination region of an examination object representing the examination region as the result of a radiological examination (e.g. MRI) according to a first measurement protocol (e.g. in the form of a T1-weighted MRI image), a synthetic representation representing the examination region as the result of a radiological examination (e.g. MRI) according to a second measurement protocol (e.g. in the form of a T2-weighted MRI image), the first measurement protocol and the second measurement protocol being different.

A “machine learning model” can be understood as a computer-implemented data processing architecture. The model is able to receive input data and to supply output data on the basis of said input data and model parameters. The model can learn a relationship between the input data and the output data by means of training. During training, the model parameters can be adjusted so as to supply a desired output for a particular input.

During the training of such a model, the model is presented with training data from which it can learn. The trained machine learning model is the result of the training process. Besides input data, the training data include the correct output data (target data) that are to be generated by the model on the basis of the input data. During training, patterns that map the input data onto the target data are recognized.

In the training process, the input data of the training data are input into the model, and the model generates output data. The output data are compared with the target data. Model parameters are altered so as to reduce the differences between the output data and the target data to a (defined) minimum. The modification of model parameters in order to reduce the differences can be done using an optimization method such as a gradient descent method.

The differences can be quantified with the aid of a loss function. A loss function of this kind can be used to calculate a loss value for a given pair of output data and target data. The goal of the training process can consist in altering (adjusting) the parameters of the machine-learning model so as to reduce the loss value for all pairs of the training data set to a (defined) minimum.

For example, if the output data and the target data are numbers, the loss function can be the absolute difference between these numbers. In this case, a high absolute loss value can mean that one or more model parameters need to be altered to a substantial degree.

For example, for output data in the form of vectors, difference metrics between vectors such as the mean squared error, a cosine distance, a norm of the difference vector such as a Euclidean distance, a Chebyshev distance, an Lp norm of a difference vector, a weighted norm or any other type of difference metric between two vectors can be chosen as the loss function.

In the case of higher-dimensional outputs, such as two-dimensional, three-dimensional or higher-dimensional outputs, an element-by-element difference metric can for example be used. Alternatively or in addition, the output data may be transformed into for example a one-dimensional vector before calculation of a loss value.

In the present case, the machine-learning model is trained by means of training data to generate a synthetic representation of an examination region of an examination object in a second state on the basis of at least one representation of the examination region of the examination object in a first state.

The training data comprise a set of input data and target data for each examination object of a multiplicity of examination objects.

The term “multiplicity” means at least ten, preferably more than one hundred.

Each set of input data and target data includes at least one input representation of an examination region of the examination object in a first state as input data. Each set of input data and target data also includes a target representation of the examination region of the examination object in a second state and a transformed target representation as target data.

The examination region is normally the same for all examination objects.

The transformed target representation represents at least part of the examination region of the examination object in the second state.

The transformed target representation represents at least part of the examination region in a different space compared to the target representation.

For example, the transformed target representation may represent at least part of the examination region of the examination object in frequency space if the target representation represents the examination region of the examination object in real space.

For example, the transformed target representation may represent at least part of the examination region of the examination object in real space if the target representation represents the examination region of the examination object in frequency space.

For example, the transformed target representation may represent at least part of the examination region of the examination object in projection space if the target representation represents the examination region of the examination object in real space.

For example, the transformed target representation may represent at least part of the examination region of the examination object in real space if the target representation represents the examination region of the examination object in projection space.

For example, the transformed target representation may represent at least part of the examination region of the examination object in Hough space if the target representation represents the examination region of the examination object in real space.

For example, the transformed target representation may represent at least part of the examination region of the examination object in real space if the target representation represents the examination region of the examination object in Hough space.

The transformed target representation in frequency space can be generated from a target representation in real space by Fourier transform, for example.

The transformed target representation in real space can be generated from a target representation in frequency space by inverse Fourier transform, for example.

The transformed target representation in projection space can be generated from a target representation in real space by Radon transform, for example.

The transformed target representation in Hough space can be generated from a target representation in real space by Hough transform, for example.

The training of the machine-learning model involves feeding, for each examination object of the multiplicity of examination objects, the at least one input representation of the examination region to the machine-learning model. The model generates a synthetic representation of the examination region on the basis of the at least one input representation of the examination region and on the basis of model parameters.

The synthetic representation represents the examination region preferably in the same space as the target representation.

If the at least one input representation fed to the model represents the examination region in real space, the synthetic representation preferably (but not necessarily) also represents the examination region in real space.

If the at least one input representation fed to the model represents the examination region in frequency space, the synthetic representation preferably (but not necessarily) also represents the examination region in frequency space.

If the at least one input representation fed to the model represents the examination region in projection space, the synthetic representation preferably (but not necessarily) also represents the examination region in projection space.

Generated from or in relation to the synthetic representation is a transformed synthetic representation.

The transformed synthetic representation represents the examination region preferably in the same space as the transformed target representation.

If the synthetic representation represents the examination region in real space in a second state, the transformed synthetic representation may represent at least part of the examination region in the second state in frequency space or in projection space. If the synthetic representation represents the examination region in the second state in frequency space, the transformed synthetic representation may represent at least part of the examination region in the second state in real space. If the synthetic representation represents the examination region in the second state in projection space, the transformed synthetic representation may represent at least part of the examination region in the second state in real space.

The generation of the transformed synthetic representation can be achieved by transform of the synthetic representation and/or the machine-learning model may be configured and trained to generate a transformed synthetic representation on the basis of the at least one input representation.

The machine-learning model may be configured and trained to generate on the basis of the at least one input representation of the examination region a synthetic representation of the examination region that represents i) the examination region in frequency space, if the at least one representation represents the examination region in real space, or ii) the examination region in real space, if the at least one representation represents the examination region in frequency space. In other words: the machine-learning model may be configured and trained to carry out (inter alia) a transform from real space into frequency space or vice versa.

The machine-learning model may be configured and trained to generate on the basis of the at least one input representation of the examination region a synthetic representation of the examination region that represents i) the examination region in projection space, if the at least one representation represents the examination region in real space, or ii) the examination region in real space, if the at least one representation represents the examination region in projection space. In other words: the machine-learning model may be configured and trained to carry out (inter alia) a transform from real space into projection space or vice versa.

A loss function is used to quantify the differences i) between at least part of the synthetic representation and at least part of the target representation and ii) between at least part of the transformed synthetic representation and at least part of the transformed target representation.

The loss function may comprise two terms: a first term for quantifying the differences between at least part of the synthetic representation and at least part of the target representation, and a second term for quantifying the differences between at least part of the transformed synthetic representation and at least part of the transformed target representation. The terms may, for example, be added up in the loss function. The terms may be weighted differently in the loss function. The following equation gives an example of a (total) loss function L for quantifying the differences:

L=λ₁·L₁+λ₂·L₂ (eq. 1)

Here, L is the (total) loss function, L₁ is a term which represents the differences between the synthetic representation and the target representation, L₂ is a term which quantifies the differences between the transformed synthetic representation and the transformed target representation, and λ₁ and λ₂ are weight factors which, for example, can assume values between 0 and 1 and give a different weight to the two terms in the loss function. It is possible that the weight factors are kept constant or varied during the training of the machine-learning model.

Examples of loss functions that can be used to carry out the present invention are L1 loss function, L2 loss function, Lp loss function, structural similarity index measure (SSIM), VGG loss function, perceptual loss function or a combination of the aforementioned functions, or other loss functions. Further details on loss functions can be found, for example, in the scientific literature (see for example: R. Mechrez et al.: The Contextual Loss for Image Transformation with Non-Aligned Data, 2018, arXiv: 1803.02077v4; H. Zhao et al.: Loss Functions for Image Restoration with Neural Networks, 2018, arXiv: 1511.08861v3; D. Fuoli et al.: Fourier Space Losses for Efficient Perceptual Image Super-Resolution, arXiv:2106.00783v1).

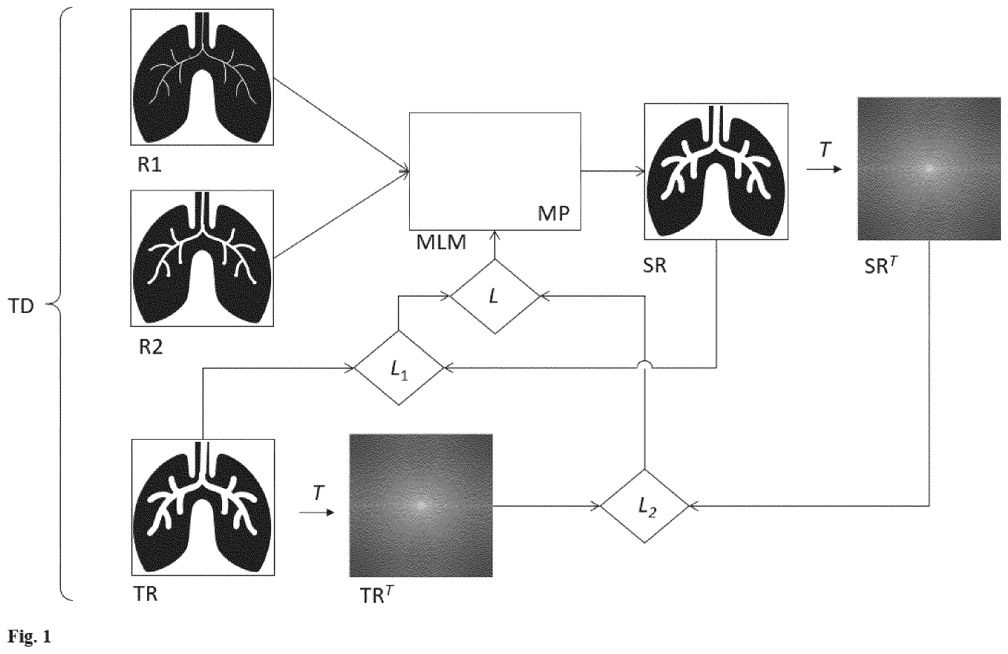

FIG. 1 shows by way of example and in schematic form the process of training the machine-learning model. The training is carried out with the aid of training data. FIG. 1 shows training data TD for an examination object. The training data TD comprise, as input data, a first input representation Ri of an examination region of the examination object and a second input representation R2 of the examination region of the examination object. The first input representation Ri and the second input representation R2 represent the examination region in real space. The first input representation R1 represents the examination region in a first state, for example without contrast agent or after the administration of a first amount of a contrast agent or in a first period of time before or after the administration of a contrast agent. The second input representation R2 represents the examination region in a second state, for example after the administration of a second amount of the contrast agent or in a second period of time after the administration of a contrast agent.

The training data TD also comprise a target representation TR as target data. The target representation TR also represents the examination region in real space. The target representation TR represents the examination region in a third state, for example after the administration of a third amount of the contrast agent or at a third moment after the administration of a contrast agent.

The machine-learning model MLM is trained to predict the target representation TR on the basis of the first input representation R1 and the second input representation R2 and on the basis of model parameters MP. The first input representation R1 and the second input representation R2 are fed to the machine-learning model as input data. The machine-learning model is configured to generate a synthetic representation SR.

In the example shown in FIG. 1, what is generated from the synthetic representation SR by means of a transform T (e.g. a Fourier transform) is a transformed synthetic representation SR^{T}. Analogously, what is generated from the target representation TR by means of the transform T is a transformed target representation TR^{T}. The transformed synthetic representation SR^{T} and the transformed target representation TR^{T} are frequency-space depictions of the examination region in the third state.

A first loss function L₁ is used for quantifying the differences between the synthetic representation SR and the target representation TR. A second loss function L₂ is used for quantifying the differences between the transformed synthetic representation SR^{T} and the transformed target representation TR^{T}. The loss values calculated by means of the loss functions L₁ and L₂ are combined in a total loss function L (e.g. by addition with or without weighting) to give a total loss value.

Model parameters MP are modified with respect to reducing the total loss value. The total loss value can be reduced with the aid of an optimization method, for example with the aid of a gradient descent method.

The process is repeated for a multiplicity of examination objects until the total loss value has reached a predefined (desired) minimum and/or until the total loss value cannot be reduced further by modifying model parameters.

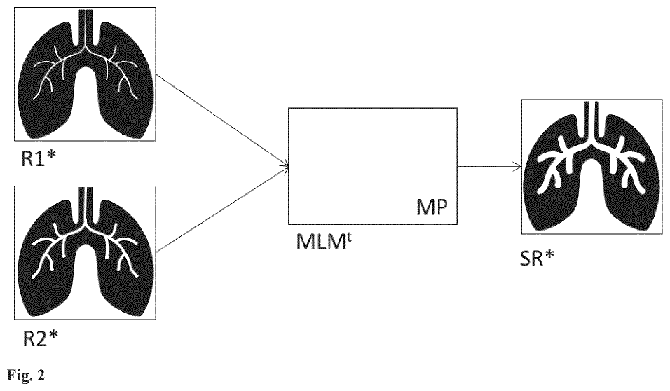

The trained machine-learning model can then be used for prediction. This is shown by way of example and in schematic form in FIG. 2: At least one input representation (R1*, R2*) of the examination region of a new examination object is fed to the trained machine-learning model MLM^{t}. The trained machine-learning model MLM^{t} generates a synthetic representation SR* of the examination region of the new examination object. Here, the term “new” means that input data from the new examination object has normally not already been used during the training and/or validation of the machine-learning model. The synthetic representation SR* generated may be output, stored and/or transmitted to a separate computer system.

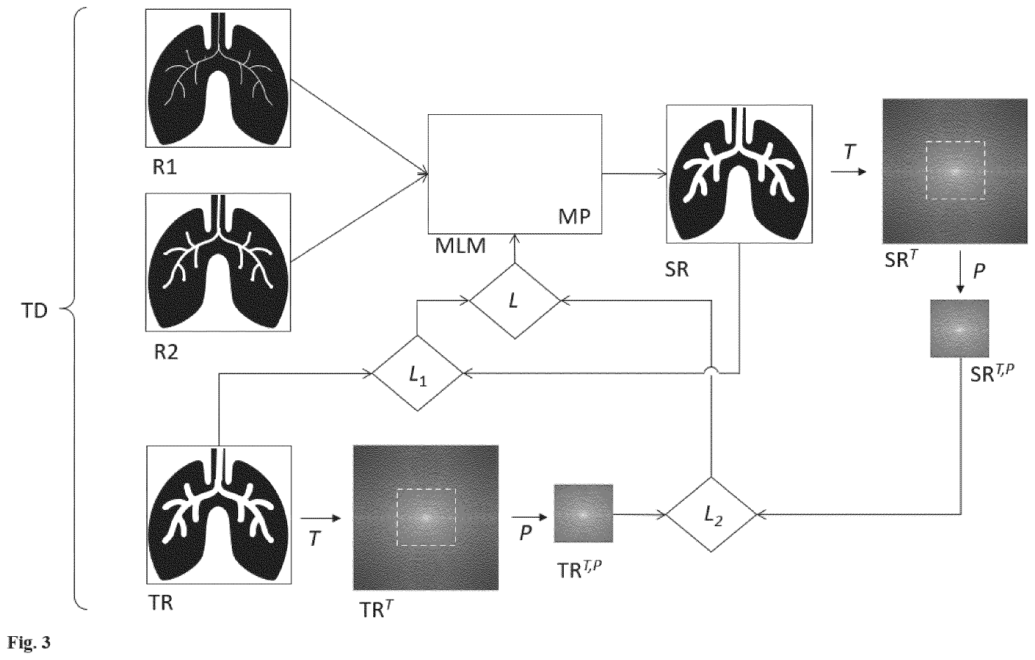

In one embodiment of the present disclosure, the quantification of the differences between the transformed synthetic representation and the transformed target representation is done only on the basis of portions of said representations. This is shown by way of example and in schematic form in FIG. 3.

The process shown in FIG. 3 corresponds to the process shown in FIG. 1, with the following differences:
The transformed target representation TR^{T} is reduced to a defined portion TR^{T,P}. The representation TR^{T,P} is part of the transformed target representation TR. The representation TR^{T,P} may be generated by a function P from the transformed target representation TR^{T}, for example by zeroing all values of frequencies outside the predefined part.
Analogously, the transformed synthetic representation SR^{T} is reduced to a defined portion SR^{T,P}.
The portions to which the transformed target representation TR^{T} and the transformed synthetic representation SR^{T} are reduced are normally mutually corresponding, i.e. they normally concern the same frequencies. In the process shown in FIG. 3, both the transformed target representation TR^{T} and the transformed synthetic representation SR^{T} have each been reduced to a portion containing the low frequencies in a region (e.g. rectangle or square) around the center (see the dashed white frames). Encoded in this region is mainly contrast information.

Therefore, in the example shown in FIG. 3, the total frequency space is not considered in the calculation by means of the loss function L₂. The representations in frequency space are reduced to a region of low frequencies; the higher frequencies are discarded. Encoded in the region of low frequencies is mainly contrast information, whereas encoded in the region of higher frequencies is mainly information about fine structures. This means that, in addition to generating the synthetic representation SR, the machine-learning model in the example shown in FIG. 3 is trained to correctly reproduce in particular the low frequencies. Thus the training focuses on contrast information.

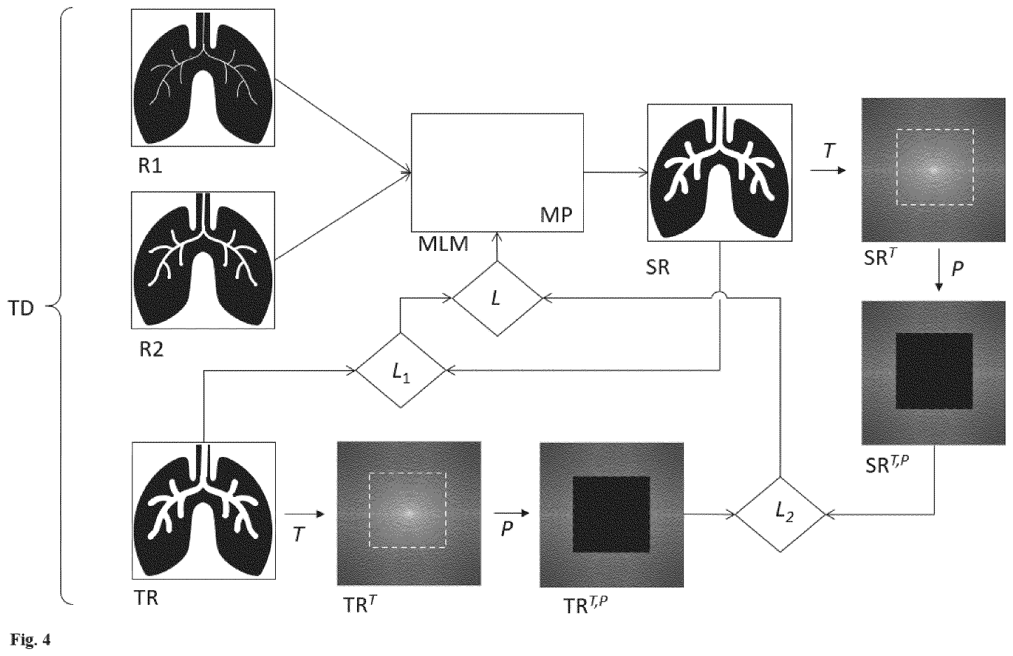

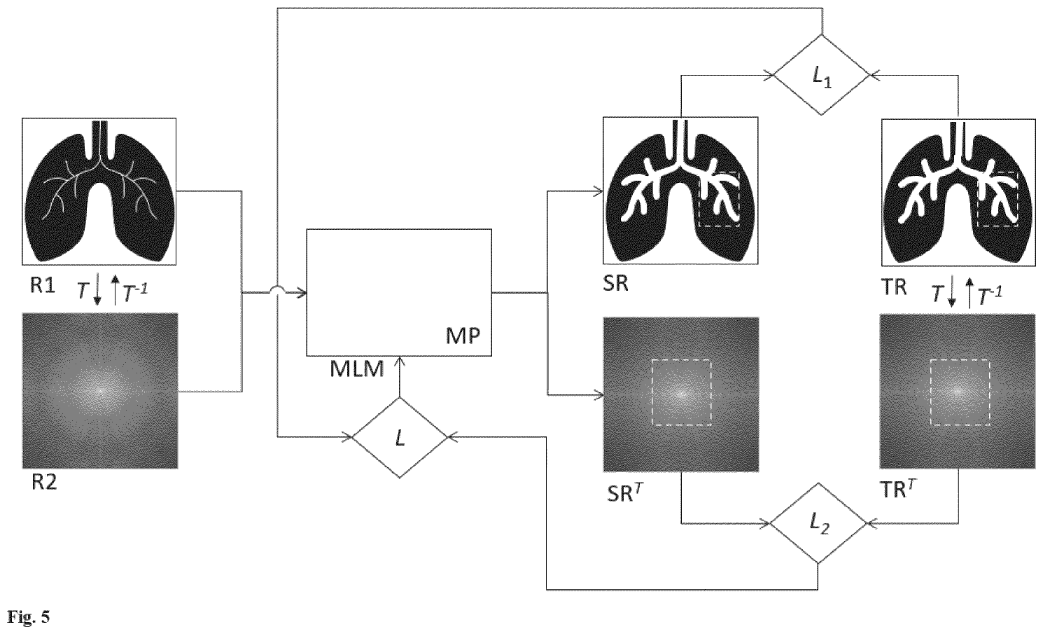

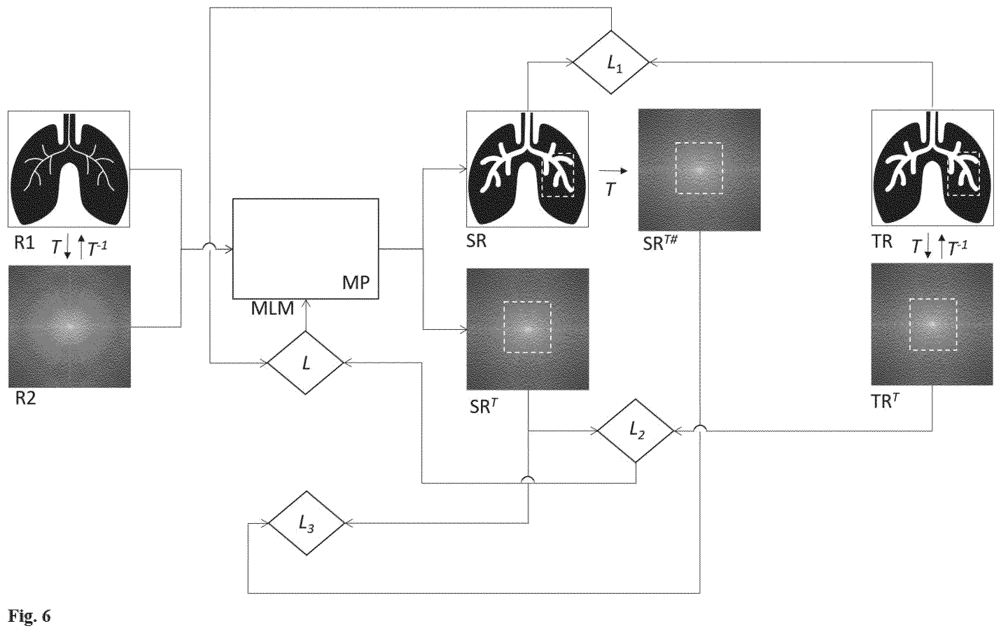

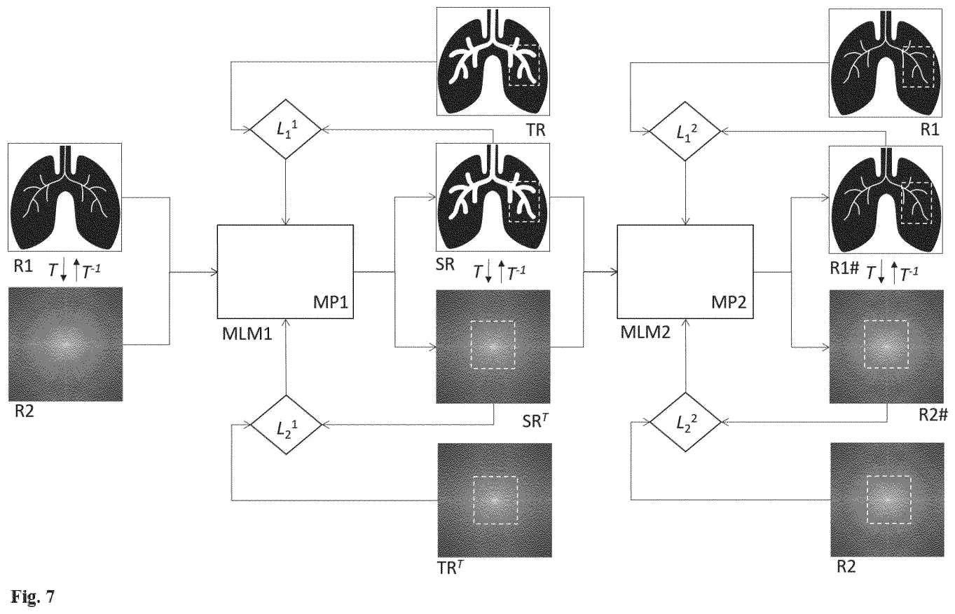

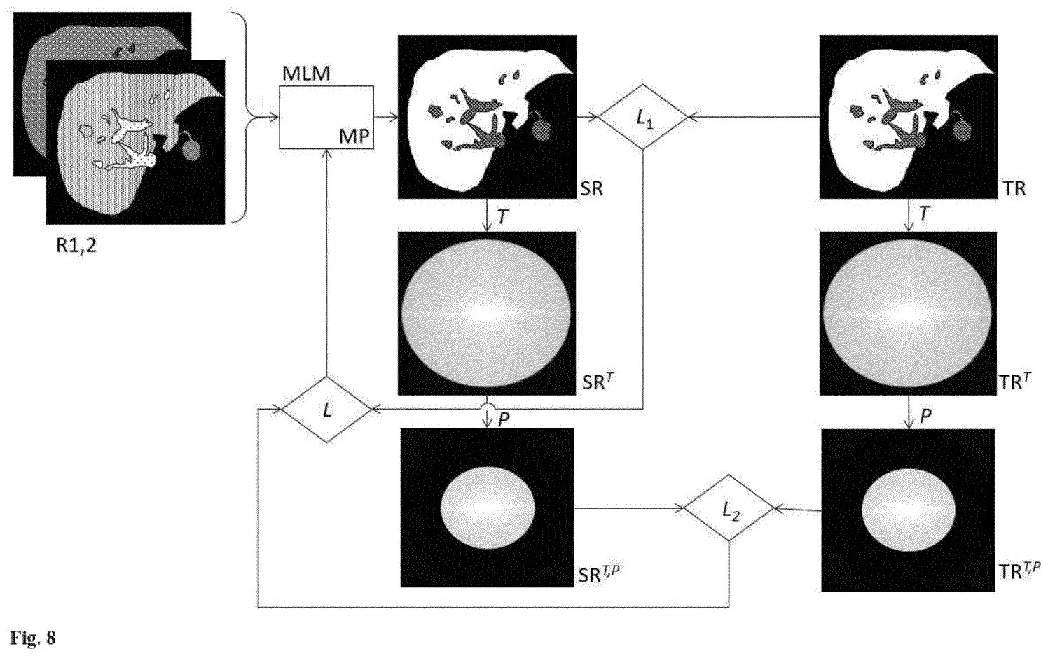

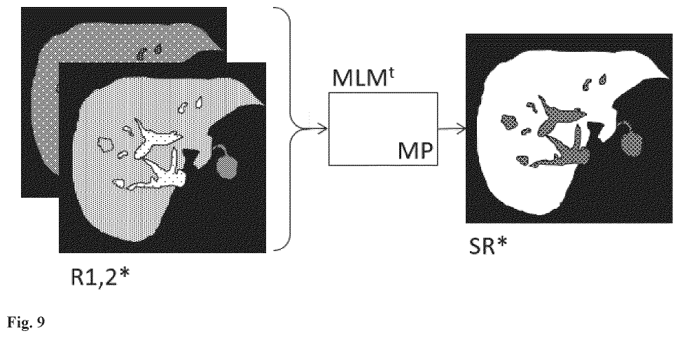

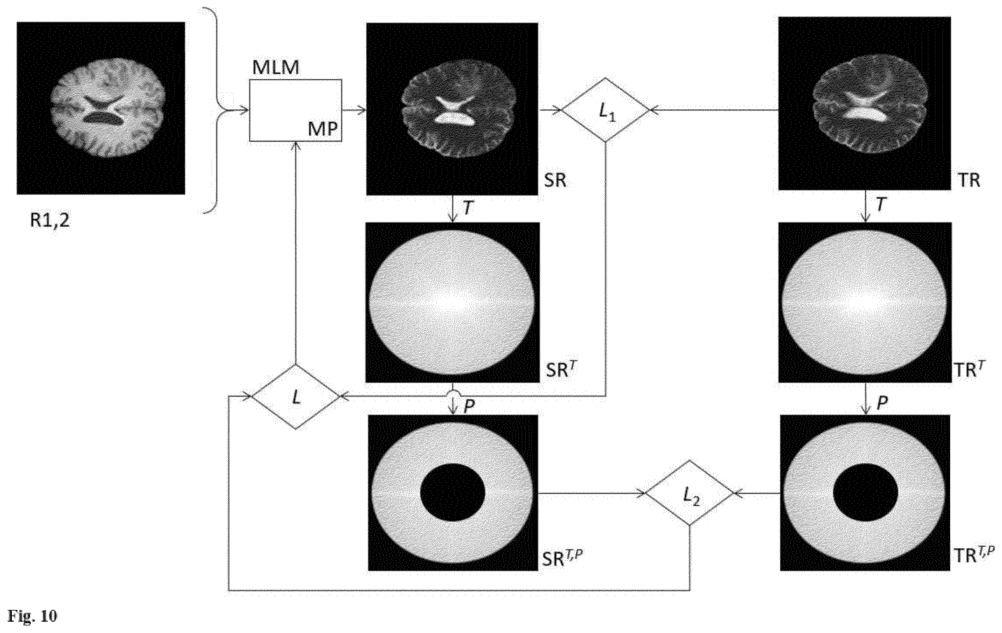

It should be noted that FIG. 3 is not to be understood as meaning that the transformed target representation TR^{T} and the transformed synthetic representation SR^{T} must be cropped to reduce them to the defined portion TR^{T,P} and the defined portion SR^{T,P}, respectively. It is also possible that the quantification of the differences between the portion SR^{T,P} and the portion TR^{T,P} by means of the loss function L₂ is achieved by only taking into account the regions SR^{T,P} and TR^{T,P} within the representations SR^{T} and TR^{T}. The term “reduce” is thus to be understood as meaning that the determination of the differences between the transformed synthetic representation SR^{T} and the transformed target representation TR^{T} is achieved by only taking into account the regions SR^{T,P} and TR^{T,P} within the representations SR^{T} and TR^{T}. This also applies analogously to FIG. 4, FIG. 5, FIG. 6, FIG. 7, FIG. 8, FIG. 10 and FIG. 11.

FIG. 4 shows another example in which the total frequency space is not considered and in which a defined frequency range is focused on instead. The process shown in FIG. 4 corresponds to the process shown in FIG. 3, with the difference that the function P ensures that the transformed target representation TR^{T} and the transformed synthetic representation SR^{T} are each reduced to a portion comprising higher frequencies, whereas low frequencies are discarded (the low frequency values are zeroed in the present example, the value of zero being represented by the color black). This means that, in addition to generating the synthetic representation SR, the machine-learning model in the example shown in FIG. 4 is trained to correctly reproduce in particular the higher frequencies and that the focus is thus on the correct reproduction of fine structures. As already explained in relation to FIG. 3, the low frequency values do not necessarily have to be zeroed; it is also possible that the quantification of the differences between the transformed synthetic representation SR^{T} and the transformed target representation TR^{T} by means of the loss function L₂ involves only taking into account the nonblack regions. This also applies analogously to FIG. 8, FIG. 10 and FIG. 11.

It should also be noted that the part to which the frequency space is reduced for the loss calculation may also assume shapes and sizes other than those shown in FIG. 3 and FIG. 4. Furthermore, multiple parts may be selected and the frequency-space depictions may be limited (reduced) to multiple parts (regions). For example, it is possible to divide the frequency space into various regions and to generate, for multiple regions or all the regions, frequency-space depictions of the examination region that each contain only the frequencies of the respective region. This makes it possible for different frequency ranges to be weighted differently/taken into account differently during training.

In the examples shown in FIG. 3 and FIG. 4, the representations R1, R2, TR and SR are representations in real space. It is also possible that one or more of these representations are frequency-space depictions and/or projection-space depictions and/or representations in one or more further/other spaces.

If the transformed synthetic representation SR^{T} and the transformed target representation TR^{T} are representations in real space, a part (or multiple parts) in the representations may likewise be selected and the representations may be reduced to said part(s). In such a case, the loss calculation thus focuses on features in real space that are found in the selected part(s).

FIG. 5, FIG. 6, FIG. 7, FIG. 8 and FIG. 9 show further embodiments of the training method.

In the embodiment shown in FIG. 5, the training data comprise for each examination object at least one input representation R1 of an examination region in a first state and a target representation TR of the examination region in a second state. The at least one input representation R1 may, for example, be the result of a first radiological examination method (e.g. MRI or CT) (i.e. one or more MRI images or one or more CT images); the target representation TR may, for example, be the result of a second radiological examination method (e.g. CT or MRI) (i.e. a CT image or an MRI image). The first radiological examination method and the second radiological examination method are normally different. It is also possible that the at least one input representation R1 is the result of a radiological examination method (e.g. MRI) according to a first measurement protocol (e.g. a T1-weighted MRI image) and that the target representation TR is the result of the radiological examination method (e.g. MRI) according to a second measurement protocol (e.g. a T2-weighted MRI image). It is also possible that the at least one input representation R1 represents an examination region without contrast agent and/or after the administration of a first amount of a contrast agent and optionally further amounts of a contrast agent, whereas the target representation TR represents the examination region after the administration of a second amount or a further amount of the contrast agent. It is also possible that the at least one input representation R1 represents an examination region in a first period of time before or after the administration of a contrast agent, and the target representation TR represents the examination region in a second period of time after the administration of a contrast agent. Further options are conceivable.

The at least one input representation R1 and the target representation TR may each be a representation in real space or a representation in frequency space. In the example shown in FIG. 5, the input representation R1 and the target representation TR are representations in real space. The machine-learning model MLM is trained to predict the target representation TR on the basis of the at least one input representation R1. A transform T is used to generate a transformed input representation R2 on the basis of the input representation R1. Analogously, the transform T is used to generate a transformed target representation TR^{T} on the basis of the target representation TR. In the example shown in FIG. 5, the transformed input representation R2 and the transformed target representation TR^{T} are representations of the examination region in frequency space. The transformed input representation R2 represents the examination region (such as the input representation R1) in the first state; the transformed target representation TR^{T} represents the examination region (such as the target representation TR) in the second state.

FIG. 5 shows that the input representation R1 can be obtained by an inverse transform T¹ on the basis of the transformed input representation R2. Analogously, a target representation TR can be obtained by the inverse transform T¹ on the basis of the transformed target representation TR^{T}. The inverse transform T¹ is the inverse transform in relation to the transform T, and this is indicated by the superscript index −1. The reference to the inverse transform is intended to make it clear that the training data must contain at least an input representation (R1 or R2) and a target representation (TR or TR^{T}); the other input representation (R2 or R1) and the other target representation (TR^{T} or TR) can be obtained from the existing representation by transform or inverse transform. This is generally applicable and does not only apply to the embodiment shown in FIG. 5.

In the embodiment shown in FIG. 5, both the input representation R1 and the transformed input representation R2 are fed to the machine-learning model MLM. The machine-learning model MLM is configured to generate both a synthetic representation SR and a transformed synthetic representation SR^{T}.

A first loss function L₁ is used to quantify the differences between at least part of the synthetic representation SR and at least part of the target representation TR. A second loss function L₂ is used to quantify the differences between at least part of the transformed synthetic representation SR^{T} and at least part of the transformed target representation TR^{T}. The dashed white frames in the transformed representations in FIG. 5 are intended to express that loss calculation by means of the loss function does not have to be based on the entire frequency-space depiction as described in relation to FIG. 3 and FIG. 4 and that, instead, the transformed representations can be reduced to a frequency range (or multiple frequency ranges). Analogously, the real-space depictions SR and TR can be reduced to one part (or multiple parts) for loss calculation (also shown by the dashed white frames). This also applies analogously to the other embodiments and does not only apply to the embodiment shown in FIG. 5.

The loss values calculated by means of the loss functions L₁ and L₂ are combined in a total loss function L (e.g. by addition with or without weighting) to give a total loss value.

Model parameters MP are modified with respect to reducing the total loss value. This can be done by means of an optimization method, for example a gradient descent method.

The process is repeated for a multiplicity of examination objects until the total loss value has reached a predefined (desired) minimum.

The synthetic representation SR and the transformed synthetic representation SR^{T} must be interconvertible just like the target representation TR and the transformed target representation TR^{T}. It is possible to introduce a further loss function that assesses the quality of such a conversion. It is thus possible to generate a transformed synthetic representation from the synthetic representation SR by the transform T and to quantify the differences between this transformed synthetic representation generated by transform and the transformed synthetic representation generated by the machine-learning model by means of a third loss function L₃. Alternatively or additionally, it is possible to generate a synthetic representation from the transformed synthetic representation SR^{T} by the inverse transform T¹ and to quantify the differences between this synthetic representation generated by inverse transform and the synthetic representation generated by the machine-learning model by means of a third loss function L₃. This is shown schematically in FIG. 6 using the example of generating a transformed synthetic representation SR^{T#} from the synthetic representation SR by transform T. In the example shown in FIG. 6, the loss function L₃ quantifies the differences between the transformed synthetic representation SR^{T#} generated by transform and the transformed synthetic representation SR^{T} generated by the machine-learning model MLM. The loss functions L₁, L₂ and L₃ are combined in a total loss function L (e.g. by addition with or without weighting). In the total loss function L, the individual terms may have different weights, and the weights may be kept constant or varied over the course of the training.

A possible modification of the embodiments shown in FIG. 5 and FIG. 6 is that the machine-learning model MLM is not fed the two input representations R1 and R2, but just one of the two representations. The machine-learning model may be configured and trained to generate the other of the two representations.

FIG. 7 shows in schematic form a further embodiment of the training method. In this embodiment, two machine-learning models are used: a first model MLM1 and a second model MLM2. The first machine-learning model MLM1 is trained to predict a target representation TR and/or a transformed target representation TR^{T} from an input representation R1 and/or R2. The second machine-learning model MLM2 is trained to predict the original input representation R1 and/or R2 again (to reconstruct it/them) from a predicted target representation SR and/or a predicted transformed target representation SR^{T}. The machine-learning models thus perform a cycle which can improve the quality of prediction by the first model MLM1 (which is used for later prediction).

The first machine-learning model MLM1 is fed at least one input representation R1 and/or at least one transformed input representation R2 as input data. The first model MLM1 is configured to generate a synthetic representation SR and/or a transformed synthetic representation SR^{T} on the basis of the input data and model parameters MP1. A transformed synthetic representation SR^{T} may also be generated by transform T of the synthetic representation SR. A synthetic representation SR may also be generated by inverse transform T¹ of the transformed synthetic representation SR^{T}.

Differences between the synthetic representation SR and the target representation TR may be quantified by means of a loss function L₁¹. Differences between the transformed synthetic representation SR^{T} and the transformed target representation TR^{T} may be quantified by means of a loss function L₂¹.

Differences between a synthetic representation SR obtained by inverse transform T¹ and the synthetic representation SR generated by the model MLM1 may be quantified by means of a loss function L₃¹ (not shown in FIG. 7). Alternatively or additionally, differences between a transformed synthetic representation SR^{T} obtained by transform T and the transformed synthetic representation SR^{T} generated by the model MLM1 may be quantified by means of a loss function L₄¹ (not shown in FIG. 7).

The loss functions L₁¹, L₂¹ and, if present, L₃¹ and/or L₄¹ may be combined to form a total loss function (not shown in FIG. 7).

Model parameters MP1 may be modified with respect to reducing the total loss value.

The synthetic representation SR and/or the transformed synthetic representation SR^{T} is/are fed to the second machine-learning model MLM2. The second model MLM2 is configured to reconstruct (predict) the first input representation R1 and/or the second input representation R2. The second model MLM2 is configured to generate a predicted first input representation R1# and/or a predicted second input representation R2# on the basis of the synthetic representation SR and/or the transformed synthetic representation SR^{T} and on the basis of model parameters MP2. A second input representation R2# may also be generated by transform T of the first input representation R1# and/or a first input representation R1# may also be generated by inverse transform T¹ of the second input representation R2#.

Differences between the predicted first input representation R1# and the first input representation R1 may be quantified by means of a loss function L₁². Differences between the predicted second input representation R2# and the second input representation R2 may be quantified by means of a loss function L₂².

Differences between an input representation obtained by inverse transform T¹ and the input representation generated by the model MLM2 may be quantified by means of a loss function L₃² (not shown in FIG. 7). Alternatively or additionally, differences between an input representation obtained by transform and the input representation generated by the model MLM2 may be quantified by means of a loss function L₄² (not shown in FIG. 7).

The loss functions L₁², L₂² and, if present, L₃² and/or L₄² may be combined to form a total loss function (not shown in FIG. 7).

Model parameters MP2 may be modified with respect to reducing the total loss value.

In the examples shown in FIGS. 1, 3, 4, 5, 6 and 7, the transformed representations are representations of the examination region in frequency space. It should be noted that the representations may also be representations in a different space, for example representations in projection space and/or in Hough space and/or in another space. It should also be noted that the machine-learning model may be trained by means of various (more than two) transformed representations. For example, there may be not only a transformed target representation in frequency space and a synthetic representation in frequency space, but also a transformed target representation and a transformed synthetic representation in projection space, in Hough space and/or in another/further space.

FIG. 8 shows in schematic form a further example of a method for training a machine-learning model. FIG. 9 shows the use of the trained machine-learning model for prediction. The example shown in FIG. 8 and FIG. 9 is concerned with quickening a magnetic resonance imaging examination of the liver of an examination object using a hepatobiliary contrast agent.

An example of a hepatobiliary contrast agent is the disodium salt of gadoxetic acid (Gd-EOB-DTPA disodium), which is described in U.S. Pat. No. 6,039,931A and is commercially available under the trade names Primovist® and Eovist®. Further hepatobiliary contrast agents are described inter alia in WO2022/194777. A further MRI contrast agent having lower uptake into hepatocytes is gadobenate dimeglumine (Multihance®).

A hepatobiliary contrast agent can be used for the detection of tumors in the liver. Blood is supplied to healthy liver tissue primarily via the portal vein (vena portae), whereas most primary tumors are supplied by the liver artery (arteria hepatica). After intravenous injection of a bolus of contrast agent, it is accordingly possible to observe a time delay between the signal increase in the healthy liver parenchyma and in the tumor.

Besides malignant tumors, what are commonly found in the liver are benign lesions such as cysts, hemangiomas and focal nodular hyperplasias (FNH). A proper planning of therapy requires that these be differentiated from the malignant tumors. Primovist® can be used for the identification of benign and malignant focal liver lesions. By means of T1-weighted MRI, it provides information about the character of said lesions. Differentiation is achieved by utilizing the difference in blood supply to liver and tumor and the time profile of contrast enhancement.

In the case of the contrast enhancement achieved by Primovist® during the wash-in phase, what are observed are typical perfusion patterns which provide information for the characterization of the lesions. Depicting the vascularization helps to characterize the lesion types and to determine the spatial relationship between tumor and blood vessels.

In the case of T1-weighted MRI images, Primovist® leads, 10-20 minutes after the injection (in the hepatobiliary phase), to a distinct signal enhancement in the healthy liver parenchyma, whereas lesions containing no hepatocytes or only a few hepatocytes, for example metastases or moderately to poorly differentiated hepatocellular carcinomas (HCCs), appear as darker regions.

Tracking the spread of the contrast agent over time thus provides a good way of detecting and differentially diagnosing focal liver lesions; however, the examination extends over a comparatively long period of time. Over this period of time, movements by the patient should be largely avoided in order to minimize movement artifacts in the MRI images. The lengthy restriction of movement can be unpleasant for a patient.

Accordingly, it is already proposed in WO2021/052896A1 that MRI images of the liver of a patient during the hepatobiliary phase not be generated by measurement, but instead be predicted on the basis of MRI images from one or more preceding phases.

FIG. 8 and FIG. 9 show an improved approach compared to the method described in WO2021/052896A1:

The machine-learning model MLM is trained on the basis of training data. The training data comprise, for each examination object of a multiplicity of examination objects, one or more input representations R1,2 of the liver or part of the liver during a first period of time before and/or after the administration of a hepatobiliary contrast agent and at least one target representation TR of the liver or part of the liver during a second period of time after the administration of the hepatobiliary contrast agent.

The input representation(s) R1,2 and the target representation TR are normally generated using an MRI scanner. The hepatobiliary contrast agent may, for example, be administered as a bolus into an arm vein of the examination object in a dose based on body weight.

The input representation(s) R1,2 may represent the liver or part of the liver during the native phase, arterial phase, portal venous phase and/or transitional phase (as described in WO2021/052896A1 and the references cited therein).

The target representation shows the liver or part of the liver during the hepatobiliary phase, i.e. for example 10 to 20 minutes after the administration of the hepatobiliary contrast agent.

The input representation(s) R1,2 and the target representation TR shown in FIG. 8 are representations of the liver or part of the liver in real space. When using multiple input representations R1,2, the input representations may represent the examination region in different states, namely at different moments in the native phase, arterial phase, portal venous phase and/or transitional phase. The target representation TR represents the examination region in a further state, namely at a moment in the hepatobiliary phase.

The input representations R1,2 (with or without prior preprocessing, which may include, for example, motion correction, coregistration and/or color space conversion) are passed to a machine-learning model MLM.

The machine-learning model is configured to generate a synthetic representation SR on the basis of the input representation(s) R1,2 and on the basis of model parameters MP.

What is generated from the synthetic representation SR by means of a transform T (e.g. a Fourier transform) is a transformed synthetic representation SR^{T}.

The transformed synthetic representation SR^{T} is, for example, a representation of the liver or part of the liver during the hepatobiliary phase in frequency space.

The transformed synthetic representation SR^{T} is reduced by means of a function P to a predefined portion, generating a partial transformed synthetic representation SR^{T,P}. The function P reduces the transformed synthetic representation SR^{T} to a predefined frequency range. In the present example, frequencies outside a circle of a defined radius around the origin of the transformed synthetic representation SR^{T} are zeroed, such that only the frequencies inside the circle remain. In other words, the higher frequencies in which fine-structure information is encoded are deleted, and what remain are the low frequencies in which contrast information is encoded.

An analogous procedure is used for the target representation TR:

What is generated from the target representation TR by means of a transform T (e.g. a Fourier transform) is a transformed target representation TR^{T}. The transformed target representation TR^{T} is a representation of the liver or part of the liver during the hepatobiliary phase in frequency space. The transformed target representation TR^{T} is reduced by means of the function P to a predefined portion, generating a partial transformed target representation TR^{T,P}. The function P2 reduces the transformed target representation TR^{T} to the same frequency range as the transformed synthetic representation SR^{T}. In the present example, frequencies outside a circle of a defined radius around the origin of the transformed target representation TR^{T} are zeroed, such that only the frequencies inside the circle remain. In other words, the higher frequencies in which fine-structure information is encoded are deleted, and what remain are the low frequencies in which contrast information is encoded.

The quality of prediction by the machine-learning model is assessed by calculating a loss value by means of a loss function L. In the present example, the loss function L is composed of two terms: a first loss function L₁ and a second loss function L₂.

The first loss function L₁ quantifies the differences between the synthetic representation SR and the target representation TR.

The second loss function L₂ quantifies the differences between the partial transformed synthetic representation SR^{T} and the partial transformed target representation TR^{T}.

The terms for L₁ and L₂ may, for example, be provided with weight factors and added up in the total loss function L, as shown in the equation eq. 1 above.

In an optimization method, for example a gradient descent method, the model parameters MP can be modified with respect to reducing the loss value calculated by means of the total loss function L.

The process described is repeated for the other examination objects of the multiplicity of examination objects. The training can be ended when the loss values calculated by means of the total loss function L reach a defined minimum, i.e. the quality of prediction reaches a desired level.

FIG. 9 shows the use of the trained machine-learning model for prediction. The model may have been trained as described in relation to FIG. 8. The trained machine-learning model MLM^{t} is fed one or more input representations R1,2* of the liver or part of the liver of a new examination object. Here, the term “new” means that the input representations R1,2* have not already been used in training the model.

The input representation(s) R1,2* represent(s) the liver or part of the liver of the new examination object before and/or after the administration of a hepatobiliary contrast agent, which for example may have been administered in the form of a bolus into an arm vein of the new examination object.

The input representation(s) R1,2* represent(s) the liver or part of the liver of the new examination object in the native phase, arterial phase, portal venous phase and/or transitional phase.

The input representation(s) R1,2* represent(s) the liver or part of the liver of the new examination object in real space.

The trained machine-learning model is configured and trained to predict a synthetic representation SR* on the basis of the input representation(s) R1,2*.

The synthetic representation SR* represents the liver or part of the liver during the hepatobiliary phase, i.e. for example 10 to 20 minutes after the administration of the hepatobiliary contrast agent.

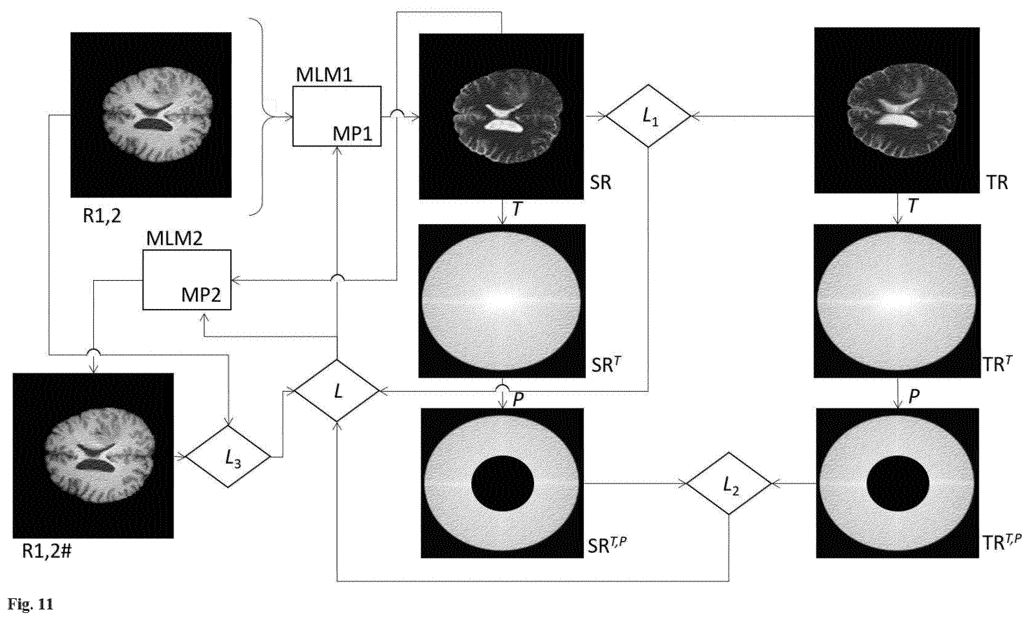

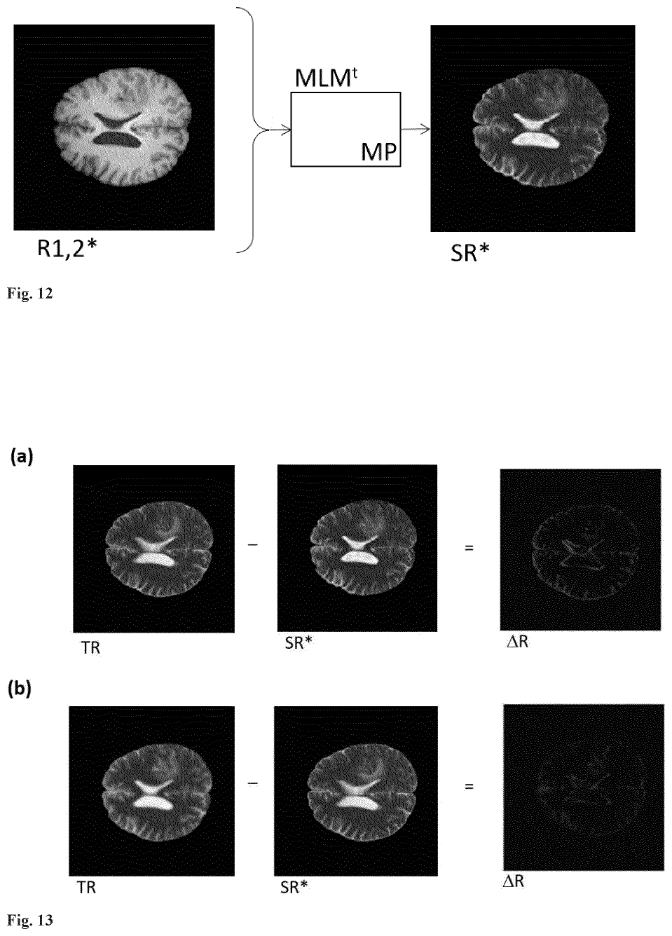

FIG. 10, FIG. 11 and FIG. 12 show in schematic form further examples of the method for training the machine-learning model and for use of the trained machine-learning model for prediction. FIG. 10 and FIG. 11 each show the training method; FIG. 12 shows the prediction method.

In the example shown in FIG. 10, the machine-learning model MLM is trained to generate, on the basis of one or more input representation(s) R1,2 representing an examination region of an examination object before and/or after the administration of a first amount of a contrast agent and on the basis of model parameters MP, a synthetic representation SR of the examination region of the examination object, the synthetic representation SR representing the examination region after the administration of a second amount of the contrast agent, the second amount being larger than the first amount. In other words, the machine-learning model is trained to predict a representation of the examination region after the administration of an amount of contrast agent larger than the amount actually administered. A target representation TR represents the examination region of the examination object after the administration of the second (larger) amount of the contrast agent.

In the present example, the examination region is a brain of a human. The at least one input representation R1,2, the target representation TR and the synthetic representation SR represent the brain in real space.

The at least one input representation R1,2 is input into the machine-learning model MLM. The machine-learning model generates the synthetic representation SR.

A transform T is used to transform the synthetic representation SR into a transformed synthetic transformation SR^{T}. The transformed synthetic transformation SR^{T} is reduced with the aid of a function P to one part; what is formed is a partial transformed synthetic transformation SR^{T,P}.

Analogously, generated from the target representation SR by means of the transform T is a transformed target representation TR^{T}, and generated by means of the function P from the transformed target representation TR^{T} is a partial transformed target representation TR^{T,P}.

The transformed synthetic representation SR^{T} and the transformed target representation TR^{T} represent the examination region in frequency space. The partial transformed synthetic representation SR^{T,P} is a transformed synthetic representation SR^{T} reduced to a frequency range of higher frequencies. The partial transformed target representation TR^{T,P} is a transformed target representation TR^{T} reduced to the frequency range of higher frequencies. The machine-learning model MLM is thus trained to focus on fine structure information encoded in the higher frequencies.

The machine-learning model is trained with the aid of a loss function L. The loss function L quantifies the differences i) between the synthetic representation SR and the target representation TR by means of a first term L₁ and ii) between the partial transformed synthetic representation SR^{T,P} and the partial transformed target representation TR^{T,P} by means of a second term L₂. In the loss function, the first term L₁ and the second term L₂ may, for example, each be multiplied by a weight factor followed by addition of the resulting products (as described by the equation eq. 1).

In an optimization method, for example a gradient descent method, the model parameters MP can be modified with respect to reducing the loss value calculated by means of the function L.

The process described is repeated for other examination objects. The training can be ended when the loss values calculated by means of the loss function L reach a defined minimum, i.e. the quality of prediction reaches a desired level.

FIG. 11 shows the training method discussed in relation to FIG. 10, having the following augmentations/changes:
In the training method, two machine-learning models are used: a first model MLM1 and a second model MLM2.
The first machine-learning model MLM1 executes the functions described in relation to FIG. 10 for the machine-learning model MLM.
The second machine-learning model is configured and trained to reproduce the at least one input representation R1,2. In other words, the second machine-learning model is configured and trained to generate a predicted input representation R1,2 on the basis of the synthetic representation SR and on the basis of model parameters MP2.
The loss function L has a third term L₃ which quantifies the differences between the input representation R1,2 and the predicted input representation R1,2#.
In an optimization method, for example a gradient descent method, the model parameters MP1 und MP2 can be modified with respect to reducing the loss value calculated by means of the function L.

FIG. 12 shows the use of the machine-learning model MLM or MLM1 trained according to FIG. 10 or FIG. 11, the trained model being identified by MLM^{t} in FIG. 12.

The trained machine-learning model MLM^{t} is fed at least one input representation R1,2* representing the brain of a new examination object. The term “new” means that the at least one input representation R1,2* has not already been used in training. The at least one input representation R1,2* represents the brain of the new examination object before and/or after the administration of a first amount of a contrast agent.

The trained machine-learning model generates, on the basis of the at least one input representation R1,2*, a synthetic representation SR* representing the brain after the administration of a second amount of the contrast agent.

FIG. 13 shows the result of validation (a) of the machine-learning model trained according to FIG. 10 and (b) of the machine-learning model trained according to FIG. 11. Each of the models was fed at least one input representation of a new examination object, as described in relation to FIG. 12. Each of the models respectively generates a synthetic representation SR*. A target representation TR exists for the examination object. FIG. 13 shows the generation of a differential representation; for each model, a differential representation AR is generated by subtraction of the respective synthetic representation SR* from the target representation TR. In the case of a perfect model, each of the synthetic representations SR* corresponds to the target representation TR and the differential representation AR is completely black (all values are zero). It can be seen in FIG. 13 that, in the case of the machine-learning model trained according to FIG. 10 (FIG. 13 (a)), more pixels differ from zero than in the case of the machine-learning model trained according to FIG. 11 (FIG. 13 (b)). Thus, in the present example, the quality of prediction by the machine-learning model trained according to FIG. 11 is higher than the quality of prediction by the machine-learning model trained according to FIG. 10.

A further embodiment of the present disclosure relates to the use of an MRI contrast agent in a CT examination method. The use of an MRI contrast agent in a computed tomography examination is described for example in J. Thomas et al.: Gadoxetate disodium enhanced spectral dual-energy CT for evaluation of cholangiocarcinoma: Preliminary data, Annals of Medicine and Surgery, 2016, 6. 10.1016/j.amsu.2016.01.001.

In a CT examination, MRI contrast agents usually have a lower contrast-enhancing effect than CT contrast agents. However, it can be advantageous to employ an MRI contrast agent in a CT examination. An example is a minimally invasive intervention in the liver of a patient, where a surgeon is monitoring the procedure by means of a CT scanner. The advantage of computed tomography (CT) over magnetic resonance imaging is that surgical procedures in an examination region of an examination object are possible while generating CT images of the examination region. Thus, while performing a procedure in the examination region, a surgeon will be able to visualize the examination region by CT and to follow the procedure on a monitor.

For example, if a surgeon wishes to perform a procedure in a patient's liver in order for example to carry out a biopsy on a liver lesion or to remove a tumor, the contrast between a liver lesion or tumor and healthy liver tissue will not be as pronounced in a CT image of the liver as it is in an MRI image after administration of a hepatobiliary contrast agent. There are currently no known and/or authorized CT-specific hepatobiliary contrast agents in CT. The use of an MRI contrast agent, more particularly a hepatobiliary MRI contrast agent, in computed tomography thus combines the possibility of differentiating between healthy and diseased liver tissue and the possibility of carrying out an operation with simultaneous visualization of the liver.

In magnetic resonance imaging (MRI), superparamagnetic substances or paramagnetic substances are normally used as contrast agents. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), gadobutrol (Gadovist®), and gadoxetic acid (Primovist®/Eovist®).

The contrast agents most commonly used in MRI are paramagnetic contrast agents based on gadolinium. These agents are usually administered via an intravenous (i.v.) bolus injection.

From their pattern of distribution in tissue, gadolinium-based contrast agents can be roughly divided into extracellular and intracellular contrast agents.

Extracellular contrast agents refer to low-molecular-weight, water-soluble compounds that, after intravenous administration, are distributed to the blood vessels and the interstitial spaces. After a certain, comparatively short period of circulation in the bloodstream, they are excreted via the kidneys. Extracellular MRI contrast agents include, for example, the gadolinium chelates gadobutrol (Gadovist®), gadoteridol (Prohance®), gadoteric acid (Dotarem®), gadopentetic acid (Magnevist®) and gadodiamide (Omnican®).

Intracellular contrast agents are taken up into the cells of tissues to a certain extent and subsequently excreted. Intracellular MRI contrast agents based on gadoxetic acid are distinguished for example in that they undergo a degree of specific uptake by liver cells (hepatocytes), accumulate in the functional tissue (parenchyma) and enhance contrasts in healthy liver tissue, before being subsequently excreted via the gallbladder into the feces. Examples of such contrast agents based on gadoxetic acid are described in U.S. Pat. No. 6,039,931A; they are commercially available for example under the trade names Primovist® and Eovist®. A further MRI contrast agent having a lower uptake into the hepatocytes is gadobenate dimeglumine (Multihance®). Contrast agents that undergo at least a degree of uptake by liver cells are also referred to as hepatobiliary contrast agents.

The MRI contrast agent can be an intracellular or extracellular contrast agent. The MRI contrast agent can also be a mixture of more than one (e.g. two) contrast agents.

In one embodiment, the MRI contrast agent is an intracellular, preferably hepatobiliary, contrast agent. In one embodiment, the contrast agent used is a substance or a substance mixture having gadoxetic acid or a salt of gadoxetic acid as contrast-enhancing active substance. For example, it may be the disodium salt of gadoxetic acid (Gd-EOB-DTPA disodium), also known as gadoxetate disodium (GD). GD is approved at a dose of 0.1 ml/kg body weight (BW) (0.025 mmol/kg BW Gd). The recommended administration of GD comprises an undiluted intravenous bolus injection at a flow rate of about 2 ml/second, followed by flushing of the i.v. cannula with a physiological saline solution.

The machine-learning model is trained using training data, the training data comprising a set of input data and target data for each examination object of a multiplicity of examination objects,
each set comprising
   at least one input representation of an examination region of the examination object in a first state as input data and
   a target representation of the examination region of the examination object in a second state and a transformed target representation as target data.

The at least one input representation is at least one CT image. The at least one CT image represents the examination region before and/or after the administration of the MRI contrast agent.

The target representation may be, for example, an MRI image representing the examination region after administration of the MRI contrast agent.

The at least one input representation may be, for example, at least one representation in real space and/or in projection space.

The target representation may be, for example, a representation in real space (e.g. if the at least one input representation is one or more representations in real space in projection space). The target representation may also be a representation of the examination region in frequency space.

The transformed target representation may be a representation in real space, in projection space or in frequency space. The transformed target representation represents the examination region in a different space compared to the target representation.

The machine-learning model is configured and trained to generate on the basis of at least one input representation of the examination region and model parameters a synthetic representation of the examination region of the examination object.

The training comprises for each examination object of the multiplicity of examination objects:
feeding the at least one input representation of the examination region of the examination object to the machine-learning model,
receiving a synthetic representation of the examination region of the examination object from the machine-learning model,
generating and/or receiving a transformed synthetic representation on the basis of the synthetic representation and/or in relation to the synthetic representation, the transformed synthetic representation representing at least part of the examination region of the examination object in a different space compared to the synthetic representation,
quantifying the differences i) between at least part of the synthetic representation and at least part of the target representation and ii) between at least part of the transformed synthetic representation and at least part of the transformed target representation by means of a loss function,
reducing the differences by modifying model parameters.

The synthetic representation represents the examination region in the same space as the target representation, and the transformed synthetic representation represents the examination region in the same space as the transformed target representation.

When the differences have reached a predefined minimum or the differences cannot be reduced further by modifying the model parameters, the training can be ended.

The trained machine-learning model and/or the model parameters may be output, stored and/or transmitted to a separate computer system.

The trained machine-learning model can be used for generation of a synthetic representation of the examination region of an examination object. To this end, at least one new input representation of the examination region of a new examination object in the first state is received.

Here, the term “new” means that data from the new examination object has normally not been used during the training and/or validation of the machine-learning model.

The at least one input representation is at least one CT image representing the examination region of the new examination object before and/or after the administration of the MRI contrast agent.

The at least one input representation of the examination region of the new examination object represents the examination region in the same space as the input representations used in training the machine-learning model.

The at least one input representation of the examination region of the new examination object is input into the trained machine-learning model.

The trained machine-learning model generates a synthetic representation of the examination region of the new examination object in the second state. The synthetic representation is a synthetic CT image preferably in real space. The synthetic representation represents the examination region of the examination object, for example after the administration of the MRI contrast agent.

The synthetic representation may be output and/or stored and/or transmitted to a separate computer system.

The synthetic representation shows regions of the examination region containing MRI contrast agents relative to the surrounding tissue having higher contrast than the at least one input representation.

Further embodiments of the present disclosure are:

1. A method for training a machine-learning model, the method comprising:
receiving and/or providing training data, the training data comprising a set of input data and target data for each examination object of a multiplicity of examination objects,
each set comprising
   at least one input representation of an examination region of the examination object in a first state as input data and
   a target representation of the examination region of the examination object in a second state and a transformed target representation as target data,
the transformed target representation representing at least part of the examination region of the examination object
   in frequency space, if the target representation represents the examination region of the examination object in real space, or
   in real space, if the target representation represents the examination region of the examination object in frequency space,
training a machine-learning model, the machine-learning model being configured to generate on the basis of at least one input representation and model parameters a synthetic representation of the examination region of the examination object,
wherein the training comprises for each examination object of the multiplicity of examination objects:
   feeding the at least one input representation to the machine-learning model,
   receiving a synthetic representation of the examination region of the examination object from the machine-learning model,
   generating and/or receiving a transformed synthetic representation on the basis of and/or in relation to the synthetic representation, the transformed synthetic representation representing at least part of the examination region of the examination object
      in frequency space, if the synthetic representation represents the examination region of the examination object in real space, or
      in real space, if the synthetic representation represents the examination region of the examination object in frequency space,
   quantifying the differences i) between at least part of the synthetic representation and at least part of the target representation and ii) between at least part of the transformed synthetic representation and at least part of the transformed target representation by means of a loss function,
   modifying model parameters in respect of reduced differences,
outputting and/or storing the trained machine-learning model and/or the model parameters and/or transmitting the trained machine-learning model and/or the model parameters to a separate computer system.
   2. The method according to embodiment 1,
wherein the receiving and/or providing of training data comprises:
   generating a partial transformed target representation, the partial transformed target representation being reduced to one part or multiple parts of the transformed target representation
wherein the generating and/or receiving of a transformed synthetic representation on the basis of and/or in relation to the synthetic representation comprises:
   generating a partial transformed synthetic representation, the partial transformed synthetic representation being reduced to one part or multiple parts of the transformed synthetic representation,
wherein the quantifying of the differences between the transformed synthetic representation and the transformed target representation comprises:
   quantifying the differences between the partial transformed synthetic representation and the partial transformed target representation.
      3. The method according to embodiment 1 or 2,
wherein the training comprises for each examination object of the multiplicity of examination objects:
   feeding the at least one input representation to the machine-learning model,
   receiving the synthetic representation and a first transformed synthetic representation of the examination region of the examination object from the machine-learning model, the first transformed synthetic representation representing at least part of the examination region of the examination object
      in frequency space, if the synthetic representation represents the examination region of the examination object in real space, or
      in real space, if the synthetic representation represents the examination region of the examination object in frequency space,
   generating a second transformed synthetic representation on the basis of the synthetic representation by means of a transform, the second transformed synthetic representation representing at least part of the examination region of the examination object
      in frequency space, if the synthetic representation represents the examination region of the examination object in real space, or
      in real space, if the synthetic representation represents the examination region of the examination object in frequency space,
   quantifying the differences i) between at least part of the synthetic representation and at least part of the target representation, ii) between at least part of the first transformed synthetic representation and at least part of the transformed target representation and iii) between at least part of the first transformed synthetic representation and at least part of the second transformed synthetic representation by means of a loss function,
   modifying model parameters in respect of reduced differences.
      4. The method according to any of embodiments 1 to 3,
wherein the machine-learning model comprises a first machine-learning model and a second machine-learning model,
wherein the first machine-learning model is configured to generate on the basis of at least one input representation and model parameters a synthetic representation of the examination region of the examination object,
wherein the second machine-learning model is configured to reconstruct on the basis of the synthetic representation of the examination region of the examination object the input representation,
wherein the training comprises for each examination object of the multiplicity of examination objects:
   generating a transformed input representation on the basis of the input representation by means of a transform, the transformed input representation representing at least part of the examination region of the examination object
      in frequency space, if the input representation represents the examination region of the examination object in real space, or
      in real space, if the input representation represents the examination region of the examination object in frequency space,
   feeding the at least one input representation to the first machine-learning model,
   receiving a synthetic representation of the examination region of the examination object from the first machine-learning model,
   generating and/or receiving a transformed synthetic representation on the basis of and/or in relation to the synthetic representation, the transformed synthetic representation representing at least part of the examination region of the examination object
      in frequency space, if the synthetic representation represents the examination region of the examination object in real space, or
      in real space, if the synthetic representation represents the examination region of the examination object in frequency space,
   feeding the synthetic representation and/or the transformed synthetic representation to the second machine-learning model,
   receiving a predicted input representation from the second machine-learning model,
   generating and/or receiving a transformed predicted input representation on the basis of and/or in relation to the predicted input representation, the transformed predicted input representation representing at least part of the examination region of the examination object
      in frequency space, if the predicted input representation represents the examination region of the examination object in real space, or
      in real space, if the predicted input representation represents the examination region of the examination object in frequency space,
   quantifying the differences i) between at least part of the synthetic representation and at least part of the target representation, ii) between at least part of the transformed synthetic representation and at least part of the transformed target representation, iii) between at least part of the input representation and at least part of the predicted input representation and iv) between at least part of the transformed input representation and at least part of the transformed predicted input representation by means of a loss function,
   modifying model parameters in respect of reduced differences.
      5. The method according to any of embodiments 1 to 4, wherein the input representation represents the examination region before and/or after the administration of a first amount of contrast agent, and the synthetic representation represents the examination region after the administration of a second amount of contrast agent, the first amount being different from, preferably lower than, the second amount.
      6. The method according to any of embodiments 1 to 5, wherein the input representation represents the examination region in a first period of time before and/or after the administration of an amount of a contrast agent and wherein the synthetic representation represents the examination region in a second period of time after the administration of the amount of the contrast agent, the second period of time following the first period of time.
      7. The method according to any of embodiments 1 to 6, wherein the input representation represents the examination region before and/or after the administration of an amount of a first contrast agent, and the synthetic representation represents the examination region after the administration of a second amount of a second contrast agent, the first contrast agent and the second contrast agent being different.
      8. The method according to any of embodiments 1 to 7, wherein the input representation represents the examination region in a first radiological examination, and the synthetic representation represents the examination region in a second radiological examination, one of the radiological examinations being an MRI examination and the other radiological examination being a CT examination.
      9. The method according to any of embodiments 1 to 8, wherein the examination region is a liver or part of a liver of a human.
      10. The method according to any of embodiments 1 to 9, wherein each input representation of the at least one input representation is a representation of the examination region in real space, the target representation is a representation of the examination region in real space, and the synthetic representation is a representation of the examination region in real space.
      11. The method according to any of embodiments 2 to 9, wherein the partial transformed synthetic representation represents the examination region in frequency space, the partial transformed synthetic representation being reduced to a frequency range of the transformed synthetic representation, contrast information being encoded in the frequency range.
      12. The method according to any of embodiments 2 to 19, wherein the partial transformed synthetic representation represents the examination region in frequency space, the partial transformed synthetic representation being reduced to a frequency range of the transformed synthetic representation, information about fine structures being encoded in the frequency range.
      13. A computer-implemented method for generating a synthetic representation of an examination region of an examination object, the method comprising:
receiving at least one input representation of the examination region of the examination object in a first state,
providing a trained machine-learning model,
   the trained machine-learning model having been trained by means of training data to generate on the basis of at least one input representation of an examination region of an examination object in a first state a synthetic representation of the examination region in a second state,
   the training data comprising for each examination object of a multiplicity of examination objects i) an input representation of the examination region, ii) a target representation and iii) a transformed target representation,
   the transformed target representation representing at least part of the examination region of the examination object in frequency space, if the target representation represents the examination region of the examination object in real space, or in real space, if the target representation represents the examination region of the examination object in frequency space,
   the machine-learning model having been trained to minimize differences, for each examination object, between i) at least part of the synthetic representation and at least part of the target representation and ii) between at least part of a transformed synthetic representation and at least part of the transformed target representation,
inputting the at least one received input representation of the examination region of the examination object into the trained machine-learning model,
receiving a synthetic representation of the examination region of the examination object in the second state from the machine-learning model,
outputting and/or storing the synthetic representation and/or transmitting the synthetic representation to a separate computer system.
   14. The method according to embodiment 13, wherein the trained machine-learning model has been trained in a method according to any of embodiments 1 to 12.
   15. A computer system comprising
a receiving unit,
a control and calculation unit and
an output unit,
   wherein the control and calculation unit is configured to cause the receiving unit to receive at least one input representation of an examination region of a new examination object in a first state,
   wherein the control and calculation unit is configured to input the received at least one input representation into a trained machine-learning model,
      the trained machine-learning model having been trained by means of training data to generate on the basis of at least one input representation of an examination region of an examination object in a first state a synthetic representation of the examination region in a second state,
      the training data comprising for each examination object of a multiplicity of examination objects i) an input representation of the examination region, ii) a target representation and iii) a transformed target representation,
      the transformed target representation representing at least part of the examination region of the examination object in frequency space, if the target representation represents the examination region of the examination object in real space, or in real space, if the target representation represents the examination region of the examination object in frequency space,
      the machine-learning model having been trained to minimize differences, for each examination object, between i) at least part of the synthetic representation and at least part of the target representation and ii) between at least part of a transformed synthetic representation and at least part of the transformed target representation,
   wherein the control and calculation unit is configured to receive from the machine-learning model a synthetic representation of the examination region of the new examination object in the second state,
   wherein the control and calculation unit is configured to cause the output unit to output the synthetic representation and/or to store it and/or to transmit it to a separate computer system.
      16. A computer program product comprising a computer program that can be loaded into a working memory of a computer system, where it causes the computer system to execute the following steps:
receiving at least one input representation of an examination region of a new examination object in a first state,
inputting the at least one input representation of the examination region of the new examination object into a trained machine-learning model,
   the trained machine-learning model having been trained by means of training data to generate on the basis of at least one input representation of an examination region of an examination object in a first state a synthetic representation of the examination region in a second state,
   the training data comprising for each examination object of a multiplicity of examination objects i) an input representation of the examination region, ii) a target representation and iii) a transformed target representation,
   the transformed target representation representing at least part of the examination region of the examination object in frequency space, if the target representation represents the examination region of the examination object in real space, or in real space, if the target representation represents the examination region of the examination object in frequency space,
   the machine-learning model having been trained to minimize differences, for each examination object, between i) at least part of the synthetic representation and at least part of the target representation and ii) between at least part of a transformed synthetic representation and at least part of the transformed target representation,
receiving a synthetic representation of the examination region of the new examination object in the second state from the machine-learning model,
outputting and/or storing the synthetic representation and/or transmitting the synthetic representation to a separate computer system.

The machine-learning models according to the present disclosure may, for example, be an artificial neural network or include such an artificial neural network or a plurality thereof An artificial neural network comprises at least three layers of processing elements: a first layer with input neurons (nodes), an N-th layer with at least one output neuron (nodes), and N−2 inner layers, where N is a natural number and greater than 2.

The input neurons serve to receive the input representations. Normally, there is one input neuron for each pixel or voxel of an input representation when the representation is a real-space depiction in the form of a raster graphic, or one input neuron for each frequency present in the input representation when the representation is a frequency-space depiction. There may be additional input neurons for additional input values (for example information about the examination region, information about the examination object, information about the conditions when the input representation was generated, information about the state represented by the input representation, and/or information about the moment at which or period of time in which the input representation had been generated).

The output neurons may serve to output a synthetic representation representing the examination region in a different state.

The processing elements of the layers between the input neurons and the output neurons are connected to one another in a predetermined pattern with predetermined connection weights.

Preferably, the artificial neural network is a so-called convolutional neural network (CNN) or comprises such a network.

A CNN normally consists essentially of an alternately repeating array of filters (convolutional layer) and aggregation layers (pooling layer) terminating in one or more layers of “normal” fully connected neurons (dense/fully connected layer).

The training of the neural network may, for example, be carried out by means of a back propagation method. The goal for the network is to map the input representation(s) onto the synthetic representation as reliably as possible. The quality of prediction is described by a loss function. The goal is to minimize the loss function. In the case of the back propagation method, an artificial neural network is taught by altering the connection weights.

In the trained state, the connection weights between the processing elements contain information regarding the dynamics of the relationship between the input representation(s) and the synthetic representation that can be used to predict, on the basis of one or more representations of the examination region of a new examination object, a synthetic representation of the examination region of the new examination object. Here, the term “new” means that representations of the examination region of the new examination object have not already been used during the training of the machine-learning model.

A cross-validation method may be used to divide the data into training data sets and validation data sets. The training data set is used in the back propagation training of network weights. The validation data set is used to check the accuracy of prediction with which the trained network can be applied to unknown (new) data.

The artificial neural network may have an autoencoder architecture; for example the artificial neural network may have an architecture such as the U-Net (see for example O. Ronneberger et al.: U-net: Convolutional networks for biomedical image segmentation, International Conference on Medical image computing and computer-assisted intervention, pages 234-241, Springer, 2015, https://doi.org/10.1007/978-3-319-24574-4_28).

The artificial neural network may be a generative adversarial network (GAN) (see for example M.-Y. Liu et al.: Generative Adversarial Networks for Image and Video Synthesis: Algorithms and Applications, arXiv:2008.02793; J. Henry et al.: Pix2Pix GAN for Image-to-Image Translation, DOI: 10.13140/RG.2.2.32286.66887).

The artificial neural network may be a regularized generative adversarial network (see for example Q. Li et al.: RegGAN: An End-to-End Network for Building Footprint Generation with Boundary Regularization, Remote Sens. 2022, 14, 1835).

The artificial neural network may be a conditional adversarial network (see for example P. Isola et al.: Image-to-Image Translation with Conditional Adversarial Networks, arXiv:1611.07004 [cs.CV]).

The artificial neural network may be a transformer network (see for example D. Karimi et al.: Convolution-Free Medical Image Segmentation using Transformers, arXiv:2102.13645 [eess.IV]).

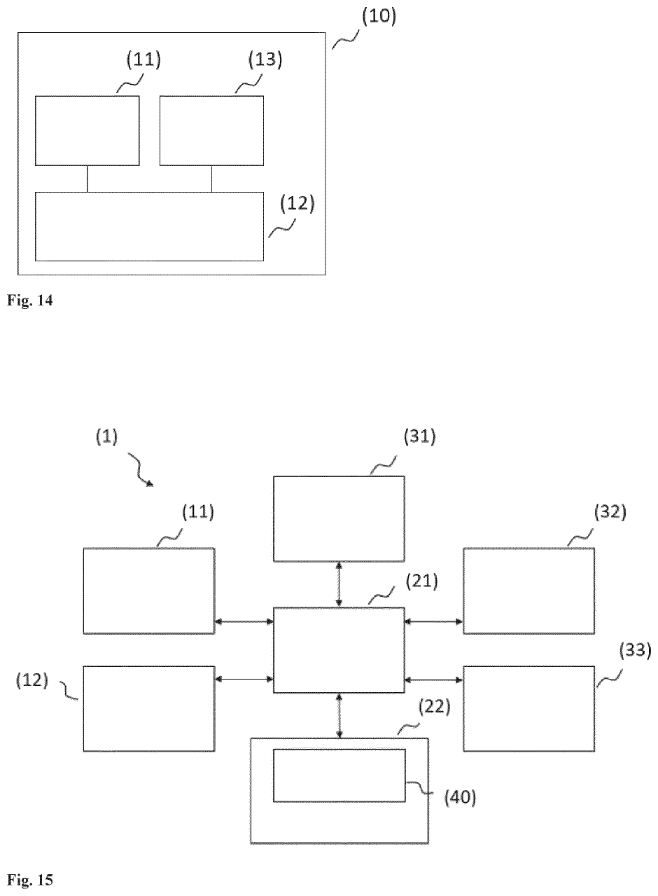

FIG. 14 shows by way of example and in schematic form a computer system according to the present disclosure, which can be used for training the machine-learning model and/or for using the trained machine-learning model for prediction.

A “computer system” is an electronic data processing system that processes data by means of programmable calculation rules. Such a system typically comprises a “computer”, which is the unit that includes a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (10) shown in FIG. 14 comprises a receiving unit (11), a control and calculation unit (12) and an output unit (13).

The control and calculation unit (12) serves for control of the computer system (10), for coordination of the data flows between the units of the computer system (10), and for the performance of calculations.

The control and calculation unit (12) is configured:
to cause the receiving unit (11) to receive at least one input representation of an examination region of a new examination object,
to input the received at least one input representation into a trained machine-learning model, the trained machine-learning model having been trained as described in this description,
to receive from the machine-learning model a synthetic representation of the examination region of the new examination object,
to cause the output unit (13) to output the synthetic representation and/or to store it and/or to transmit it to a separate computer system.

FIG. 15 shows by way of example and in schematic form a further embodiment of the computer system according to the invention.

The computer system (1) comprises a processing unit (21) connected to a memory (22). The processing unit (21) and the memory (22) form a control and calculation unit, as shown in FIG. 14.

The processing unit (21) may comprise one or more processors alone or in combination with one or more memories. The processing unit (21) may be customary computer hardware that is able to process information such as digital images, computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of the same or of a different computer system.

The memory (22) may be customary computer hardware that is able to store information such as digital images (for example representations of the examination region), data, computer programs and/or other digital information either temporarily and/or permanently. The memory (22) may comprise a volatile and/or nonvolatile memory and may be nonremovable or removable. Examples of suitable memories are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (21) may be connected not just to the memory (22), but also to one or more interfaces (11, 12, 31, 32, 33) in order to display, transmit and/or receive information. The interfaces may comprise one or more communication interfaces (32, 33) and/or one or more user interfaces (11, 12, 31). The one or more communication interfaces may be configured to send and/or receive information, for example to and/or from an MRI scanner, a CT scanner, an ultrasound camera, other computer systems, networks, data memories or the like. The one or more communication interfaces may be configured to transmit and/or receive information via physical (wired) and/or wireless communication connections. The one or more communication interfaces may comprise one or more interfaces for connection to a network, for example using technologies such as cellphone, Wi-Fi, satellite, cable, DSL, optical fiber and/or the like. In some examples, the one or more communication interfaces may comprise one or more close-range communication interfaces configured to connect devices with close-range communication technologies such as NFC, RFID, Bluetooth, Bluetooth LE, ZigBee, infrared (e.g. IrDA) or the like.

The user interfaces may include a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image or video recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces may furthermore include one or more interfaces for communication with peripherals such as printers and the like.

One or more computer programs (40) may be stored in the memory (22) and executed by the processing unit (21), which is thereby programmed to perform the functions described in this description. The retrieving, loading and execution of instructions of the computer program (40) may take place sequentially, such that an instruction is respectively retrieved, loaded and executed. However, the retrieving, loading and/or execution may also take place in parallel.

The machine-learning model according to the invention may also be stored in the memory (22).

The computer system according to the invention may be designed as a laptop, notebook, netbook and/or tablet PC; it may also be a component of an MRI scanner, a CT scanner or an ultrasound diagnostic device.

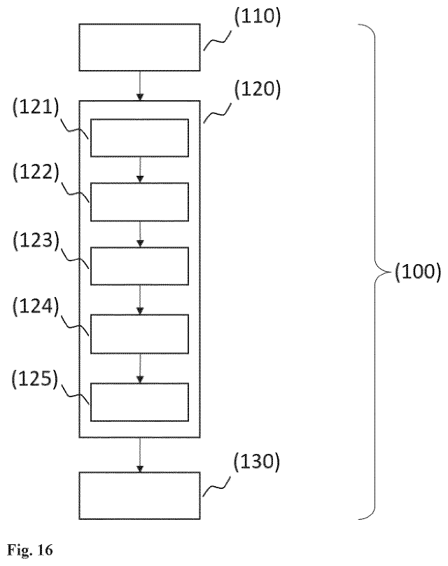

FIG. 16 shows schematically in the form of a flow chart one embodiment of the method for training a machine-learning model. The training method (100) comprises the steps of:
(110) receiving and/or providing training data, the training data comprising a set of input data and target data for each examination object of a multiplicity of examination objects,
   each set comprising
      at least one input representation of an examination region of the examination object in a first state as input data and
      a target representation of the examination region of the examination object in a second state and a transformed target representation as target data, the transformed target representation representing at least part of the examination region of the examination object in a different space compared to the target representation,
(120) training a machine-learning model, the machine-learning model being configured to generate on the basis of at least one input representation of an examination region of an examination object and model parameters a synthetic representation of the examination region of the examination object, the synthetic representation representing the examination region in the same space as the target representation,
   wherein the training comprises for each examination object of the multiplicity of examination objects:
   (121) feeding the at least one input representation to the machine-learning model,
   (122) receiving a synthetic representation of the examination region of the examination object from the machine-learning model,
   (123) generating and/or receiving a transformed synthetic representation on the basis of the synthetic representation and/or in relation to the synthetic representation, the transformed synthetic representation representing at least part of the examination region of the examination object in a different space compared to the synthetic representation, the transformed synthetic representation representing at least part of the examination region in the same space as the transformed target representation,
   (124) quantifying the differences i) between at least part of the synthetic representation and at least part of the target representation and ii) between at least part of the transformed synthetic representation and at least part of the transformed target representation by means of a loss function,
   (125) reducing the differences by modifying model parameters,
(130) outputting and/or storing the trained machine-learning model and/or the model parameters and/or transmitting the trained machine-learning model and/or the model parameters to a separate computer system and/or using the trained machine-learning model for generation of a synthetic representation of the examination region of a new examination object.

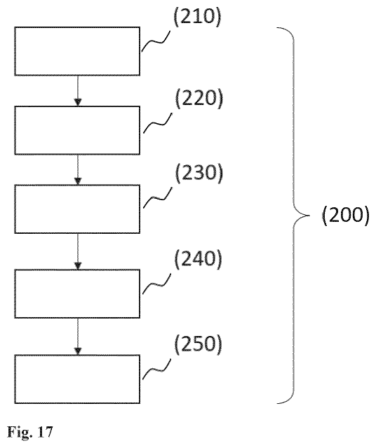

FIG. 17 shows schematically in the form of a flow chart one embodiment of the method for generating a synthetic representation of an examination region of an examination object with the aid of the trained machine-learning model. The prediction method (200) comprises the steps of
(210) providing a trained machine-learning model,
   the trained machine-learning model having been trained by means of training data to generate on the basis of at least one input representation of an examination region of an examination object in a first state a synthetic representation of the examination region in a second state,
   the training data comprising for each examination object of a multiplicity of examination objects i) an input representation of the examination region, ii) a target representation and iii) a transformed target representation,
   the at least one input representation representing the examination region of the examination object in the first state and the target representation representing the examination region of the examination object in the second state,
   the transformed target representation representing the examination region of the examination object in a different space compared to the target representation,
   the training of the machine-learning model comprising reducing differences between i) at least part of the synthetic representation and at least part of the target representation and ii) between at least part of a transformed synthetic representation and at least part of the transformed target representation, the synthetic representation representing the examination region in the same space as the target representation, the transformed synthetic representation representing the examination region in the same space as the transformed target representation,
(220) receiving at least one input representation of an examination region of a new examination object in the first state,
(230) inputting the at least one input representation of the examination region of the new examination object into the trained machine-learning model,
(240) receiving a synthetic representation of the examination region of the new examination object in the second state from the machine-learning model,
(250) outputting and/or storing the received synthetic representation and/or transmitting the synthetic representation to a separate computer system.

1. A computer-implemented method comprising:
receiving and/or providing training data (TD), the training data (TD) comprising a set of input data and target data for each examination object of a multiplicity of examination objects, each set comprising:
at least one input representation (R1, R2) of an examination region of the examination object in a first state as input data; and
a target representation (TR) of the examination region of the examination object in a second state and a transformed target representation (TR^{T}) as target data, the transformed target representation (TR^{T}) representing at least part of the examination region of the examination object in a different space compared to the target representation (TR);
training a machine-learning model (MLM), the machine-learning model (MLM) configured to generate on the basis of the at least one input representation (R1, R2) of the examination region of the examination object and model parameters (MP) a synthetic representation (SR) of the examination region of the examination object,
wherein the training comprises for each examination object of the multiplicity of examination objects:
feeding the at least one input representation (R1, R2) of the examination region of the examination object to the machine-learning model (MLM);
receiving the synthetic representation (SR) of the examination region of the examination object from the machine-learning model (MLM);
generating and/or receiving a transformed synthetic representation (SR^{T}) on the basis of the synthetic representation (SR) and/or in relation to the synthetic representation (SR), the transformed synthetic representation (SR^{T}) representing at least part of the examination region of the examination object in a different space compared to the synthetic representation (SR);
quantifying the differences:
i) between at least part of the synthetic representation (SR) and at least part of the target representation (TR); and
ii) between at least part of the transformed synthetic representation (SR^{T}) and at least part of the transformed target representation (TR^{T}) by means of a loss function (L); and
reducing the differences by modifying the model parameters (MP); and
outputting and/or storing the trained machine-learning model (MLM^{t}) and/or the model parameters (MP) and/or transmitting the trained machine-learning model (MLM^{t}) and/or the model parameters (MP) to a separate computer system and/or using the trained machine-learning model (MLM^{t}) for generation of a synthetic representation of the examination region of a new examination object.

2. The method as claimed in claim 1,
wherein the receiving and/or providing of training data (TD) comprises:
generating a partial transformed target representation (TR^{T,P}), the partial transformed target representation (TR^{T,P}) being reduced to one part or multiple parts of the transformed target representation (TR^{T}),
wherein the generating and/or receiving of the transformed synthetic representation (SR^{T}) on the basis of the synthetic representation (SR) and/or in relation to the synthetic representation (SR) comprises:
generating a partial transformed synthetic representation (SR^{T,P}), the partial transformed synthetic representation (SR^{T,P}) being reduced to one part or multiple parts of the transformed synthetic representation (SR^{T}), and
wherein the quantifying of the differences between the at least part of the transformed synthetic representation (SR^{T}) and the at least part of the transformed target representation (TR^{T}) comprises:
quantifying the differences between the partial transformed synthetic representation (SR^{T,P}) and the partial transformed target representation (TR^{T,P}).

3. The method as claimed in claim 1,
wherein the training comprises for each examination object of the multiplicity of examination objects:
feeding the at least one input representation (R1, R2) to the machine-learning model (MLM);
receiving the synthetic representation (SR) and a first transformed synthetic representation (SR^{T}) of the examination region of the examination object from the machine-learning model, the first transformed synthetic representation (SR^{T}) representing at least part of the examination region of the examination object in a different space compared to the synthetic representation (SR);
generating a second transformed synthetic representation (SR^{T#}) on the basis of the synthetic representation (SR) by means of a transform (7), the second transformed synthetic representation (SR^{T#}) representing at least part of the examination region of the examination object in the same space as the first synthetic representation (SR^{T});
quantifying the differences:
i) between at least part of the synthetic representation (SR) and at least part of the target representation (TR),
ii) between at least part of the first transformed synthetic representation (SR^{T}) and at least part of the transformed target representation (TR^{T}), and
iii) between at least part of the first transformed synthetic representation (SR^{T}) and at least part of the second transformed synthetic representation (SR^{T#}) by means of a loss function (L); and
reducing the differences by modifying the model parameters.

4. The method as claimed in claim 1,
wherein the machine-learning model (MLM) comprises a first machine-learning model (MLM1) and a second machine-learning model (MLM2),
wherein the first machine-learning model (MLM1) is configured to generate on the basis of the at least one input representation (R1, R2) and model parameters of the first machine-learning model (MP1) the synthetic representation (SR) of the examination region of the examination object,
wherein the second machine-learning model (MLM2) is configured to reconstruct on the basis of the synthetic representation (SR) of the examination region of the examination object and model parameters of the second machine-learning model (MP2) at least one input representation (R1, R2), and
wherein the training comprises for each examination object of the multiplicity of examination objects:
generating a transformed input representation (R2) on the basis of the at least one input representation (R1) by means of a transform, the transformed input representation (R2) representing at least part of the examination region of the examination object in a different space compared to the at least one input representation (R1);
feeding the at least one input representation (R1) and/or the transformed input representation (R2) to the first machine-learning model (MLM1);
receiving the synthetic representation (SR) of the examination region of the examination object from the first machine-learning model (MLM1);
generating and/or receiving the transformed synthetic representation (SR^{T}) on the basis of and/or in relation to the synthetic representation (SR), the transformed synthetic representation (SR^{T}) representing at least part of the examination region of the examination object in a different space compared to the synthetic representation (SR);
feeding the synthetic representation (SR) and/or the transformed synthetic representation (SR^{T}) to the second machine-learning model (MLM2);
receiving a predicted input representation (R1#) from the second machine-learning model (MLM2);
generating and/or receiving a transformed predicted input representation (R2#) on the basis of and/or in relation to the predicted input representation (R1#), the transformed predicted input representation (R2#) representing at least part of the examination region of the examination object in a different space compared to the predicted input representation (R1#);
quantifying the differences:
i) between at least part of the synthetic representation (SR) and at least part of the target representation (TR),
ii) between at least part of the transformed synthetic representation (SR^{T}) and at least part of the transformed target representation (TR^{T}),
iii) between at least part of the input representation (R1) and at least part of the predicted input representation (R1#); and
iv) between at least part of the transformed input representation (R2) and at least part of the transformed predicted input representation (R2#) by means of a loss function (L); and
reducing the differences by modifying model parameters.

5. The method as claimed in claim 1, wherein a) the at least one input representation (R1, R2) represents the examination region before and/or after the administration of a first amount of contrast agent, and the synthetic representation (SR) represents the examination region after the administration of a second amount of contrast agent, the first amount being different from the second amount: or b) the at least one input representation (R1, R2) represents the examination region in a first period of time before and/or after the administration of an amount of a contrast agent and wherein the synthetic representation (SR) represents the examination region in a second period of time after the administration of the amount of the contrast agent, the second period of time following the first period of time.

6. (canceled)

7. The method as claimed in claim 1, wherein a) the at least one input representation (R1, R2) represents the examination region before and/or after the administration of an amount of a first contrast agent, and the synthetic representation (SR) represents the examination region after the administration of a second amount of a second contrast agent, the first contrast agent and the second contrast agent being different; or b) the at least one input representation (R1, R2) represents the examination region in a first radiological examination, and the synthetic representation represents the examination region in a second radiological examination, one of the radiological examinations being an MRI examination and the other radiological examination being a CT examination.

8. (canceled)

9. The method as claimed in claim 1, wherein the at least one input representation (R1, R2) represents the examination region as a result of a radiological examination according to a first measurement protocol, and the synthetic representation represents the examination region as a result of a radiological examination according to a second measurement protocol, the first measurement protocol differing from the second measurement protocol.

10. The method as claimed in claim 1, wherein the at least one input representation (R1, R2) is at least one CT image representing the examination region before and/or after the administration of an MRI contrast agent and the target representation is an MRI image representing the examination region after the administration of the MRI contrast agent.

11. The method as claimed in claim 1, wherein the examination region is a liver or part of a liver of a human.

12. The method as claimed in claim 1, wherein the transformed target representation (TR^{T}) and the transformed synthetic representation (SR) represent at least part of the examination region of the examination object in frequency space, if the target representation (TR) and the synthetic representation (SR) represent the examination region of the examination object in real space, or in real space, if the target representation (TR) and the synthetic representation (SR) represent the examination region of the examination object in frequency space.

13. The method as claimed in claim 1, wherein the transformed target representation (TR^{T}) and the transformed synthetic representation (SR^{T}) represent at least part of the examination region of the examination object in projection space, if the target representation (TR) and the synthetic representation (SR) represent the examination region of the examination object in real space, or in real space, if the target representation (TR) and the synthetic representation (SR) represent the examination region of the examination object in projection space.

14. The method as claimed in claim 1, wherein each input representation of the at least one input representation (R1, R2) is a representation of the examination region in real space, the target representation (TR) is a representation of the examination region in real space, and the synthetic representation (SR) is a representation of the examination region in real space.

15. The method as claimed in claim 2, wherein the partial transformed synthetic representation (SR^{T,P}) represents the examination region in frequency space, the partial transformed synthetic representation (SR^{T,P}) being reduced to a frequency range of the transformed synthetic representation (SR^{T}), and contrast information being encoded in the frequency range.

16. The method as claimed in claim 2, wherein the partial transformed synthetic representation (SR^{T,P}) represents the examination region in frequency space, the partial transformed synthetic representation (SR^{T,P}) being reduced to a frequency range of the transformed synthetic representation (SR^{T}), and information about fine structures being encoded in the frequency range.

17. The method as claimed in claim 1, further comprising:
receiving at least one input representation (R1*, R2*) of an examination region of a new examination object in the first state;
inputting the at least one input representation (R1*, R2*) of the examination region of the new examination object into the trained machine-learning model (MLM);
receiving a synthetic representation (SR*) of the examination region of the new examination object in the second state from the machine-learning model (MLM^{t}); and
outputting and/or storing the received synthetic representation (SR*) and/or transmitting the received synthetic representation (SR*) to a separate computer system.

18. The method as claimed in claim 17, wherein the at least one input representation (R1, R2) is at least one CT image representing the examination region before and/or after the administration of an MRI contrast agent and the target representation is an MRI image representing the examination region after the administration of the MRI contrast agent.

19. The method as claimed in claim 17, wherein the at least one input representation (R1*, R2*) of the examination region of the new examination object in the first state being at least one CT image before and/or after the administration of a contrast agent and the synthetic representation (SR*) of the examination region of the new examination object in the second state being a synthetic CT image after the administration of the contrast agent.

20. A computer system comprising:
a receiving unit;
a control and calculation unit; and
an output unit,
wherein the control and calculation unit is configured to cause the receiving unit (11) to receive at least one input representation (R1*, R2*) of an examination region of a new examination object in a first state,
wherein the control and calculation unit (12) is configured to input the received at least one input representation (R1*, R2*) into a trained machine-learning model (MLM^{t});
the trained machine-learning model (MLM^{t}) having been trained by means of training data (TD) to generate on the basis of at least one input representation (R1, R2) of an examination region of an examination object in a first state a synthetic representation (SR) of the examination region in a second state;
the training data (TD) comprising for each examination object of a multiplicity of examination objects:
i) at least one input representation (R1, R2) of the examination region,
ii) a target representation (TR) of the examination region- and
iii) a transformed target representation (TR^{T});
the at least one input representation (R1, R2) representing the examination region of the examination object in the first state and the target representation (TR) representing the examination region of the examination object in the second state;
the transformed target representation (TR^{T}) representing at least part of the examination region of the examination object in a different space compared to the target representation (TR);
the training of the machine-learning model (MLI) comprising reducing differences;
i) between at least part of the synthetic representation (SR) and at least part of the target representation (TR); and
ii) between at least part of a transformed synthetic representation (SR^{T}) and at least part of the transformed target representation (TR^{T}),
wherein the control and calculation unit is configured to receive from the machine-learning model (MLM^{t}) a synthetic representation (SR*) of the examination region of the new examination object in the second state, and
wherein the control and calculation unit is configured to cause the output unit to output the received synthetic representation (SR*) and/or to store the received synthetic representation (SR*) and/or to transmit the received synthetic representation (SR*) to a separate computer system.

21. A computer program product comprising a computer program that can be loaded into a working memory of a computer system, wherein the computer program causes the computer system to execute the following:
provide a trained machine-learning model (MLM^{t});
the trained machine-learning model (MLM^{t}) having been trained by means of training data (TD) to generate on the basis of at least one input representation (R1, R2) of an examination region of an examination object in a first state a synthetic representation (SR) of the examination region in a second state;
the training data (TD) comprising for each examination object of a multiplicity of examination objects:
i) at least one input representation (R1, R2) of the examination region;
ii) a target representation (TR) of the examination region; and
iii) a transformed target representation (TR^{T});
the at least one input representation (R1, R2) representing the examination region of the examination object in the first state and the target representation (TR) representing the examination region of the examination object in the second state;
the transformed target representation (TR^{T}) representing at least part of the examination region of the examination object in a different space compared to the target representation (TR);
the training of the machine-learning model (MLM^{t}) comprising reducing differences:
i) between at least part of the synthetic representation (SR) and at least part of the target representation (TR); and
ii) between at least part of a transformed synthetic representation (SR) and at least part of the transformed target representation (TR^{T});
receive at least one input representation (R1*, R2*) of an examination region of a new examination object in the first state;
input the at least one input representation (R1*, R2*) of the examination region of the new examination object into the trained machine-learning model (MLM^{t});
receive a synthetic representation (SR*) of the examination region of the new examination object in the second state from the trained machine-learning model (MLM^{t}); and
output and/or store the received synthetic representation (SR*) and/or transmit the received synthetic representation (SR*) to a separate computer system.

22-24. (canceled)

25. The method as claimed in claim 1, wherein the contrast agent comprises one or more of the following substance:
gadoxetate disodium;
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate;
gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
a Gd³⁺ complex of a compound of formula (I) 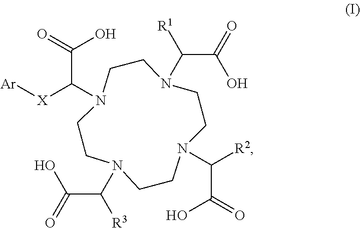
wherein:
Ar is a group selected from: 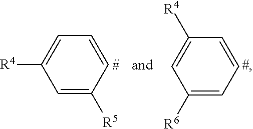
wherein _{#} is a linkage to X;
X is a group selected from:
CH₂, (CH₂)₂, (CH₂)₃, (CH₂)₄ and *—(CH₂)₂O—CH₂-^{#};
wherein * is a linkage to Ar and ^{#} is a linkage to an acetic acid residue;
R¹, R² and R³ are each independently a hydrogen atom or a group selected from C₁-C₃ alkyl, —CH₂OH, —(CH₂)₂₀H and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom,
and
R⁶ is a hydrogen atom,
or a stereoisomer, a tautomer, a hydrate, a solvate or a salt thereof, or a mixture thereof, or
a Gd³⁺ complex of a compound of formula (II) 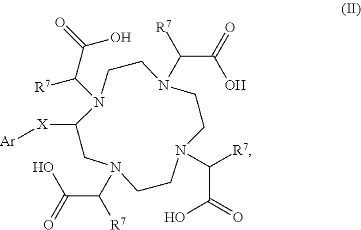
wherein:
Ar is a group selected from: 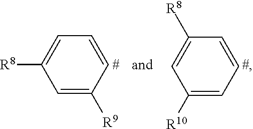
wherein _{#} is a linkage to X;
X is a group selected from CH₂, (CH₂)₂, (CH₂)₃, (CH₂)₄ and *—(CH₂)₂O—CH₂-^{#},
wherein * is a linkage to Ar and ^{#} is a linkage to the acetic acid residue;
R⁷ is a hydrogen atom or a group selected from C₁-C₃ alkyl, —CH₂OH, —(CH₂)₂OH and —CH₂OCH₃;
R⁸ is a group selected from:
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, a tautomer, a hydrate, a solvate or a salt thereof, or a mixture thereof.
